(19) United States
(12) Patent Application Publication
(10) Pub. No.: US 2024/0161927 A1
(43) Pub. Date: May 16, 2024

(54) METHODS AND APPARATUS FOR CLASSIFYING A CAPNOGRAM AND PREDICTING A CARDIORESPIRATORY DISEASE USING A CAPNOGRAM

(71) Applicant: TidaSense Limited, Cambridge (GB)

(72) Inventors: Ameera Patel, Cambridge (GB); Henry Alexander Broomfield, Cambridge (GB); Julian Charles Carter, Cambridge (GB); Ahmed Bahaaeldin Mohamed Selim, Cambridge (GB); Matthew Richard Haines, Cambridge (GB)

(21) Appl. No.: 18/503,518

(22) Filed: Nov. 7, 2023

(30) Foreign Application Priority Data
Nov. 7, 2022 (GB) 2216572.4
Oct. 20, 2023 (GB) 2316122.7
Oct. 30, 2023 (GB) 2316600.2

(51) Int. Cl. G16H 50/20; A61B 5/097

(52) U.S. Cl. CPC G16H 50/20; A61B 5/097

(57) ABSTRACT

According to examples there may be provided a method for classifying a capnogram, the method comprising: obtaining a breath waveform from a capnogram determining one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau; extracting features of the breath waveform using the transition points; and applying a trained machine learning model to the extracted features, wherein the trained machine learning model is configured to output a classification of the capnogram from the features.

### BACKGROUND

Cardiorespiratory diseases, referring to cardiovascular and respiratory diseases, are a leading cause of premature death worldwide. Cardiovascular diseases refer to conditions that affect the heart or blood circulation. Respiratory diseases, also known as lung diseases, refer to a variety of conditions that cause breathing difficulties for people with the condition. Asthma and chronic obstructive pulmonary disease (COPD) are two common examples, where COPD refers to a class of obstructive respiratory disease that primarily includes emphysema and chronic bronchitis. COPD is a progressive disease with no known cure which has been reported as the third most common cause of mortality worldwide. This has led to COPD being widely studied across the world as part of an effort to improve early diagnosis and treatment to reduce its impact on the quality of life of a patient.

The prevailing standard for COPD diagnostics is spirometry, which relies upon a patient's ability to forcefully exhale. However, a major disadvantage of this measurement is that it is effort-dependent and thus both unreliable and non-specific, particularly for patients with COPD that may be asymptomatic or have early stage disease. Capnometry is an alternative technique that can be used in assessing lung health by measuring the partial pressure of carbon dioxide in respired air. An advantage of capnometry over spirometry is that it can be used with tidal breathing and so only requires a patient to breathe as they would naturally. Capnometry produces a tidal breath record (also referred to as a capnogram) with a series of breath waveforms, and the characteristics of these waveforms can greatly vary between healthy patients and those with cardiorespiratory diseases, such as COPD, asthma and heart failure, depending on the severity of their condition.

Predicting cardiorespiratory diseases using capnometry is still in its infancy and so continued development is needed on many aspects before it can be widely adopted. For example, which characteristics and features of a capnogram are most important for classifying the capnogram and predicting cardiorespiratory diseases? How can these features be determined to help predict the presence of a cardiorespiratory disease in a patient? How can this be done quickly and efficiently in order to provide a method that is viable in the real world? The present invention provides solutions to these problems.

### SUMMARY OF INVENTION

According to a first aspect, the present disclosure provides a method for classifying a capnogram, the method comprising: obtaining a breath waveform from a capnogram; determining one or more transition points of the breath waveform, wherein the transition points comprise: a delta transition point between an expiratory baseline and an expiratory upstroke; a gamma transition point between an inspiratory downstroke and an inspiratory baseline; and, an alpha transition point between the expiratory upstroke and an expiratory plateau, extracting features of the breath waveform using the transition points; and using the extracted features, applying a trained machine learning model to the extracted features, wherein the trained machine learning model is configured to output a classification of the capnogram from the features.

The classification of the capnogram can be used for a variety of means. For example by highlighting and/or sorting capnograms for further analysis, or ranking the capnograms by a risk or importance factor associated with the capnogram. This may include predicting cardiorespiratory conditions or a healthy state to exclude such conditions, predicting a condition's severity or subtype, predicting medication responsiveness, or predicting other clinical outcomes such as probability of being hospitalised within a given time window. Using the trained machine learning model in this manner provides accurate and automated classifying of capnograms which has not previously been possible.

In some embodiments of the first aspect, the classification of the capnogram may be used to predict a cardiorespiratory disease. These may be considered a method for predicting whether a capnogram was produced from a user with a cardiorespiratory disease, where the trained machine learning model is configured to predict a cardiorespiratory disease.

That is, embodiments of the first aspect include a method for predicting whether a capnogram was produced from a user with a cardiorespiratory disease, the method comprising: obtaining a breath waveform from a capnogram; determining one or more transition points of the breath waveform, wherein the transition points comprise: a delta transition point between an expiratory baseline and an expiratory upstroke; a gamma transition point between an inspiratory downstroke and an inspiratory baseline; and, an alpha transition point between the expiratory upstroke and an expiratory plateau, extracting features of the breath waveform using the transition points; and using the extracted features, applying a trained machine learning model to the extracted features, wherein the trained machine learning model is configured to predict a cardiorespiratory disease from the features.

In particular, the method may be considered as being for predicting a cardiorespiratory disease from, or using, a capnogram, i.e. whether the capnogram was produced by a diseased respiratory tract and/or blood circulatory system (herein circulatory system). An objective is to use data yielded from a device configured to measure CO₂ concentration to build a classifier that distinguishes between capnographs that are produced by healthy lungs and capnographs that are produced by lungs or airways with a cardiorespiratory disease. For example, the cardiorespiratory disease may be COPD, pulmonary fibrosis, asthma, asthma-COPD overlap syndrome (ACOS), or small airways disease. Throughout the present application we may refer to disease. By disease we also mean subtypes of a same disease, but refer simply to disease for brevity. Predicting a cardiorespiratory disease may be considered predicting a state of health, and so includes predicting the absence of a cardiorespiratory disease, whether that refers to a healthy patient, or a patient without a cardiorespiratory disease (but having another type of disease). Predicting a cardiorespiratory disease from the features also includes predicting the type of cardiorespiratory disease, subtype of cardiorespiratory disease, and/or the severity of the cardiorespiratory disease from the features. This information may be represented by the classification of the capnogram.

The method of prediction using the trained model, and the method of training, may each be performed remotely from the patient associated with the capnogram, at a local computer or a remote computer. Steps of the method may be performed entirely by a local processing unit, remote processing unit such as in the cloud, or split between local and remote processing units.

The breath waveform may be a breath waveform or a representation of it.

In examples, the waveform is a digitally sampled signal representing a quantized amplitude signal. The signal may be encoded as an array of floating point samples, each representing an amplitude of the breath signal at a point in time. The samples may not directly correspond to the amplitude of the breath waveform but should be understood as a digital representation of it. Similarly, the waveform may be encoded in any suitable manner such as an array of binary vectors or matrices.

The transition point is the sample of the digital samples corresponding to the transition point of the waveform. The method may identify the individual sample or element in the encoding, an index of the sample, or the point in time corresponding to the sample. Each sample may generally correspond to a CO₂ value at a point in time.

The trained machine learning model was trained using the same or corresponding extracted features that have been extracted from, and based on the transition points of, other capnograms labelled as being positively or negatively associated with the cardiorespiratory disease, and in some examples the severity or subtype of the disease. For example, the trained machine learning model may have been trained using a method according to any implementation of the second aspect below. As used herein, the term machine learning is also used to refer to statistical inference. For example, a statistical inference model may be referred to as a machine learning model, and a step of training a machine learning model may refer to training a statistical inference model (or a machine learning model).

Determining the transition points of the waveform enables accurate and consistent feature extraction to be performed on different segments of the breath waveform, which results in the trained machine learning model being able to more accurately and more explainably predict whether the capnogram comprising the breath waveform was produced by a user with a cardiorespiratory disease. It has been found that the delta transition point, the gamma transition point, and the alpha transition point of a breath waveform are particularly beneficial to the accuracy of capnogram prediction.

The transition points of the waveform are parameters that are relevant to the prediction of a large variety of cardiorespiratory diseases (e.g., COPD and asthma). Therefore, these steps will not need to be repeated when predicting different cardiorespiratory diseases from an individual breath waveform, further increasing the efficiency of the method and particularly so at greater scales.

Capnograms and breath waveforms produced by different respiratory tracts can vary dramatically, as can capnograms and breath waveforms produced by the same respiratory tract at different times or under different conditions (e.g., when affected by a cardiorespiratory disease). These variations have led to difficulties when attempting to accurately analyse a capnogram quickly and/or efficiently. Automating capnogram analysis has been particularly difficult due to the substantial degree of variation between breath waveforms and the wide range of factors (such as cardiorespiratory diseases, age, weight, time of day, medication use, smoking status, location, presence of other medical diseases etc) which can contribute to these variations. However, determining the transition points of the waveform, as well as using these as the basis for feature extraction and application of the machine learning model, can be readily automated in a consistent manner to improve the efficiency of the prediction while still maintaining consistently high prediction accuracy.

As the transition points mark the transitions between different phases of the breath waveform (e.g. the alpha transition point is between the expiratory upstroke and expiratory plateau), determining any of these points also improves the interpretability of the method. That is, as well as predicting a cardiorespiratory disease from the extracted features, the trained machine learning model can also indicate which phase(s) of the breath waveform contributed to the prediction and to what extent.

The terms capnogram and breath record are used interchangeably herein to refer to a continuous capnometry measurement in a single session. For example, the user or operator stopping recording of a user's breathing with a capnometry monitor is the end of a capnogram, while starting to record it again is the beginning of another capnogram. However, capnogram and breath record may also refer to a given time period of continuous CO₂ recording (e.g. in a ventilator circuit).

The skilled person will appreciate that the inspiratory baseline and expiratory baseline may be considered the same baseline of a capnogram/breath record, i.e. a respiratory baseline or a capnogram baseline. The inspiratory baseline and the expiratory baseline are named differently in order to distinguish between (once an individual breath waveform has been isolated) the baseline adjacent the expiratory periods of the breath waveform and the baseline adjacent the inspiratory periods of the breath waveform, as well as to more clearly describe the transition points of a waveform.

According to a second aspect, there is provided a method for training a machine learning model to learn indicators of a capnogram, the method comprising, for a plurality of capnograms:
i) obtaining a breath waveform from each capnogram;
ii) determining one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau;
iii) extracting features of the breath waveform using the plurality of transition points;

obtaining a label for each capnogram indicating a capnogram classification; and, using the extracted features of the plurality of breath waveforms and the corresponding labels of the respective capnograms, training a machine learning model to learn indicators of a capnogram to create a classifying function.

The plurality of capnograms may be individual capnograms from a plurality of different users and/or multiple capnograms produced by a user at different times. A plurality of breath waveforms may also be obtained from a single capnogram of the plurality of capnograms.

Step iii) of the second aspect results in a featurised breath waveform or capnogram. Steps i) to iii) may be repeated to obtain extracted features of a plurality of breath waveforms (i.e., producing a plurality of featurised breath waveforms).

In some embodiments of the second aspect, the machine learning model is trained to learn indicators of a cardiorespiratory disease (i.e. indicators of a capnogram being produced from a user with a cardiorespiratory disease) to create a predictive function, with the capnogram classification indicating whether the capnogram was from a diseased respiratory tract and/or circulatory system (i.e. the label for each capnogram indicating whether the capnogram was from a diseased respiratory tract and/or circulatory system), and the training module creating a predictive function.

That is, embodiments of the second aspect include a method for training a machine learning model to learn indicators of a capnogram being produced from a user with a cardiorespiratory disease, the method comprising, for a plurality of capnograms:
i) obtaining a breath waveform from each capnogram;
ii) determining one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau;
iii) extracting features of the breath waveform using the plurality of transition points;
   obtaining a label for each capnogram indicating whether the capnogram was from a diseased respiratory tract and/or circulatory system; and, using the extracted features of the plurality of breath waveforms and the corresponding labels of the respective capnograms, training a machine learning model to learn indicators of a cardiorespiratory disease to create a predictive function.

The machine learning model may be trained to learn indicators of a capnogram corresponding to several different cardiorespiratory diseases. Optionally, the labels for each capnogram should indicate whether the capnogram was from a respiratory tract diseased by the cardiorespiratory disease currently being trained on, and can also indicate a subtype of the cardiorespiratory disease, the severity of the disease, medication responsiveness, and/or other clinical outcomes. For example, during training of a COPD classification model, the labels for each capnogram may indicate whether the capnogram was from a COPD-afflicted respiratory tract or a non-COPD-afflicted respiratory tract. In an example of training an asthma classification model, the labels for each capnogram may indicate whether the capnogram was from an asthmatic respiratory tract or a non-asthmatic respiratory tract. The machine learning model may also be trained to learn indicators of a capnogram produced from a healthy user without any cardiorespiratory disease. It is preferable to train the model using capnograms corresponding to healthy and diseased users in order to provide a more universally accurate function.

The first aspect and the second aspect may comprise any of the following implementations:

Preferably, the breath waveform represents a single respiratory cycle. Obtaining the breath waveform may comprise splitting the capnogram into a plurality of capnogram sections, wherein each capnogram section represents a single breath waveform corresponding to the single respiratory cycle.

The single respiratory cycle should be a full recorded breath. That is, a breath recorded from exhalation to inhalation or vice versa, with complete recording of both exhalation and inhalation. The breath waveform/respiratory cycle may also correspond to a partial breath (e.g. where the recording of capnography data was started or stopped mid-breath). Transition points may still be determined (and features can be extracted using those determined points) using the techniques of the present invention even when the breath waveform corresponds to a partial breath. However, depending on when the recording of capnography data was started or stopped, it may not be possible to determine as many transition points or extract as many features compared to a breath waveform corresponding to a full breath. Therefore, breath waveforms corresponding to partial breaths may be identified and accounted for (e.g., filtered out of the analysis). Having the breath waveform represent a single respiratory cycle reduces the amount of computation required while still providing the information required (e.g., the transition points) for the trained machine learning model to accurately predict a cardiorespiratory disease. This also facilitates further analysis such as average waveform generation, and the identification of anomalies in a capnogram.

The plurality of transition points may further comprise a beta transition point between the expiratory plateau and the inspiratory downstroke. Determining additional transition points, such as the beta transition point, enables a greater number of features to be extracted from the breath waveform and so facilitates more accurate predictions by the trained machine learning model, and/or provides a better trained machine learning model. Optionally, the waveform may be segmented into an expiratory phase and inspiratory phase based on the determined beta transition point.

Preferably, determining the plurality of transition points comprises determining a derivative of the breath waveform. The derivative of the breath waveform may be helpfully implemented in several steps of the method for a variety of reasons. Several of these implementations are discussed in detail below. For example, the derivative of the breath waveform may be used as a reference against the breath waveform in order to facilitate determining of the transition point(s). Preferably, the derivative of the breath waveform is a first order differential of the breath waveform, as the first order differential typically comprises less noise than higher order derivatives. However, higher order derivatives such as a second order differential may be more appropriate for determining the plurality of transition points depending on the breath waveform being examined. Determining the plurality of transition points may also comprise determining a plurality of derivatives of the breath waveform. For example, the first order differential of the breath waveform may be used to determine a given transition point, and the second order differential of the breath waveform used to determine a different transition point.

The derivative of the breath waveform, such as the first order differential, may also be used to identify, and optionally reject, anomalous breath waveforms, thereby preventing unnecessary processing being performed. In some implementations of the first or second aspect, the method further comprises comparing the derivative to a template; and, when the derivative is not consistent with the template, rejecting the breath waveform. The template may be a differential template or another type of template. Using the derivative to identify (and reject) anomalous breath waveforms may also comprise determining whether values of the derivative are within expected ranges.

Anomalous breath waveforms can also be identified, and optionally rejected, through other means. For example, by comparing values (e.g., at random or predetermined points) of the breath waveform itself or of the derivative to expected values or ranges. The template and these expected values are determined through empirical observations of typical physiology.

Preferably, determining the derivative of the breath waveform, such as the first order differential of the breath waveform, comprises applying a time-based smoothing filter, such as a Savitsky-Golay filter, to the breath waveform. For example, the first order differential of the breath waveform is determined in conjunction with application of a Savitsky-Golay filter. The time-based smoothing filter applies smoothing and so is particularly advantageous when used with noisy data. The Savitsky-Golay filter is also very generalisable, including the use of many window sizes and orders of polynomial fitting, and so has been found to be effective with a wide range of different breath waveforms. Other examples of smoothing filters include frequency filtering, e.g. using a wavelet or short-time Fourier filter, and moving average smoothing function.

Artefacts in breath waveforms such as hump artefacts have significant impacts on the shape of the waveform and its corresponding derivatives. In some cases, for example depending on the size and position of the artefact, this can lead to inconsistent feature extraction and less accurate predictions by the trained machine learning model, or less accurate training of the model. Accounting for any hump artefacts is of particular importance when automating the prediction method (or the training method) in order to make accurate predictions on a reasonable timescale. Hump artefacts can vary dramatically depending on the respiratory tract that produced the capnogram, however they are typically represented in a breath waveform by a sharp increase in the pCO₂ before the expiratory upstroke. The increased pCO₂ of the hump is less than that of the expiratory plateau and may remain at the increased hump level until the expiratory upstroke, may partially decrease into the expiratory upstroke, or may decrease and fully return to the pCO₂ levels of the expiratory baseline.

Therefore, it is preferred that determining the plurality of transition points comprises: identifying a hump artefact in the breath waveform and, when there is a hump artefact, accounting for the hump artefact during the determining of the one or more transition points.

The hump artefact may be accounted for through a variety of manners. For example by ignoring or removing the data associated with the hump artefact, in effect subtracting out the hump artefact, or adjusting the weighting applied to the data associated with the hump artefact when determining the one or more transition points.

Preferably, identifying the hump artefact comprises: performing peak detection to identify local minima of the breath waveform; identifying prominent minima from the local minima; identifying the maximum value of the breath waveform and/or determining the beta transition point; dividing the breath waveform into a first section not including a maximum value of the breath waveform, and a second section including the maximum value of the breath waveform and/or the beta transition point; when at least one prominent minima is identified, searching for hump artefact(s) in the first section of the breath waveform; and/or when no prominent minima are identified, using the derivative of the breath waveform to search for hump artefact(s) in the first section of the breath waveform. When the derivative of the breath waveform is a first order differential of the breath waveform, using the derivative of the breath waveform to search for/identify hump artefact(s) may comprise analysing the first order differential to identify an inflection point region of the first order differential, and comparing this inflection point region to a predetermined threshold.

The maximum value of the breath waveform refers to the maximum amplitude of the breath waveform. For example, the maximum pCO₂ value measured at a point in time during the breath waveform.

The breath waveform is divided in the time dimension. That is, values up to a certain point in time (the point bounding the first and second sections) are in the first section and values after that point in time are in the second section. The boundary between the first section and the second section may be the maximum value of the breath waveform.

Alternatively, the breath waveform may be divided such that the first section and second section are defined with respect to the beta transition point. That is, the breath waveform is divided into a first section not including the beta transition point and the second section includes the beta transition point.

It has been found that minima corresponding to hump artefacts typically have CO₂ values below a certain value. Therefore, to further reduce the computational cost, a hump artefact threshold may be implemented during the identifying of the hump artefact(s). For example, the hump artefact threshold may define a maximum CO₂ value where any minima with CO₂ values above the hump threshold are discarded or not considered during the identification of the hump artefact(s). Preferably, the hump artefact threshold is from 0.5 kPa to 4 kPa. Using a threshold within this range has been found to achieve an accurate level of hump artefact for most breath waveforms (e.g., by ignoring minima in the expiratory plateau). Most preferably, the hump artefact threshold is 2 kPa.

The first section and the second section may alternatively be defined as non-overlapping sections, where the second section includes the maximum value of the breath waveform and/or the beta transition point.

The beta transition point may be determined through various means. It has been found that a reliable and efficient method (particularly during automation) of determining the beta transition point comprises: performing peak detection to find local maxima of the breath waveform; identifying prominent maxima from the local maxima; when only a single prominent maximum is identified, determining this as a beta transition point between the expiratory plateau and the inspiratory downstroke; and, when a plurality of prominent maxima are identified, determining the most prominent maximum and defining this as the beta transition point.

It has been found through extensive data analysis that the pCO₂ value of the beta transition point typically falls within or below a range of values. Therefore, a predetermined threshold (i.e., a maxima threshold) may be defined to save further processing resources. In practice, this can be implemented by ignoring maxima with (pCO₂) values below the maxima threshold (e.g., by removing these values or setting them equal to 0 during the peak detection or identification of prominent maxima. This reduces the processing resources used by the method and reduces noise from values before the maxima threshold. For example, the maxima threshold may be set to a value from 0.04 kPa to 5 kPa. These lower and upper values for the threshold account for background pressure while reliably and accurately determining the beta transition point such that accurate and useful features can be extracted based on the determined beta transition point. Preferably, the maxima threshold may be set from 1.5 kPa and 2.5 kPa. Most preferably, the maxima threshold is 2 kPa. The maxima threshold may be predetermined or, in some implementations, the maxima threshold may be determined and/or adjusted by a machine learning model as it continues to determine beta transition points of an increasing quantity of breath waveforms. This means the machine learning model can further optimise the resource usage of the methods.

When no prominent maxima are identified, the breath waveform may be rejected as this is normally due to the breath waveform being an anomaly where extracted features are less likely to be accurate or may be unreliably interpreted by a machine learning model (that is, during prediction or training). Rejecting the breath waveform prevents these outcomes at an early stage without any further unnecessary processing.

Preferably, determining the transition points comprises determining the delta transition point, and determining the delta transition point comprises: determining the first point in time at which a first order differential of the breath waveform is above a delta threshold; and defining the first point as the delta transition point.

Similarly to the maxima threshold, the delta threshold may be a predetermined threshold, or may be determined and/or adjusted by the (trained or untrained) machine learning model. Optionally, the delta threshold is configured relative to the maximum magnitude value of the first order differential of the breath waveform. For example, the delta threshold may be from 5% to 80% of the maximum magnitude point of the first order differential. Preferably, the delta threshold is from 5 to 15% of the maximum magnitude point of the first order differential. More preferably, the delta threshold is 10% of the maximum magnitude point of the first order differential. Using these ranges for the delta threshold, and the specific value of 10%, have been found to accurately determine the delta transition point such that accurate and useful features can be extracted based on the determined delta transition point.

Preferably, determining the transition points comprises determining the gamma transition point, and determining the gamma transition point comprises: identifying the minimum value of a first order differential of the breath waveform; and, defining the gamma transition point as the first point in time after the minimum value at which the first order differential of the breath waveform is higher than a gamma threshold.

The first point in time refers to substantially the first point in time, and may be near the first point in time or, for example, a data point closely corresponding with the first point in time.

Similarly to the previously discussed thresholds, the gamma threshold may be a predetermined threshold, or may be determined and/or adjusted by the (trained or untrained) machine learning model. Optionally, the gamma threshold is configured relative to the maximum magnitude value of the first order differential of the breath waveform. For example, the gamma threshold may be from 0 to the negative of 50% of the maximum magnitude point of the first order differential. Preferably, the gamma threshold is from the negative of 2% and the negative of 10% of the maximum magnitude point of the first order differential. More preferably, the delta threshold is the negative of 5% of the maximum magnitude point of the first order differential. Using these ranges for the gamma threshold, and the specific value of −5%, have been found to accurately determine the gamma transition point such that accurate and useful features can be extracted based on the determined gamma transition point.

Preferably, determining the transition points comprises: determining the alpha transition point, wherein determining the alpha transition point comprises: identifying the maximum value of a first order differential of the breath waveform; identifying the maximum value of the breath waveform and/or determining the beta transition point; and, defining the alpha transition point as the first point in time, between the maximum value of the first order differential and the maximum value of the breath waveform and/or the beta transition point, at which the first order differential of the breath waveform is less than an alpha threshold.

It will be apparent that the alpha transition point may be determined using the maximum value of the breath waveform or using the beta transition point. These two methods may be performed individually (i.e., only one is performed in order to determine the alpha transition point while using less processing resources) or both may be performed to verify the result of the other. The beta transition point only needs to be determined at this stage if this has not already been achieved, otherwise the previously determined beta transition point may be used, or the alpha transition point may be determined without using the beta transition point. Determining the alpha transition point (and/or identifying hump artefact(s)) using the beta transition point has been found to be a more reliable method than using the maximum value of the breath waveform and is suitable for a wider range of breath waveform shapes. However, the techniques using the maximum value of the breath waveform are reliably accurate with lower computational costs associated.

Similarly to the previously discussed thresholds, the alpha threshold may be a predetermined threshold, or may be determined and/or adjusted by the (trained or untrained) machine learning model. Optionally, the alpha threshold is configured relative to the maximum magnitude value of the first order differential of the breath waveform. For example, the alpha threshold may be from 0 to 80% of the maximum magnitude point of the first order differential. Preferably, the alpha threshold may be from 10% and 20% of the maximum magnitude point of the first order differential. More preferably, the alpha threshold is 15% of the maximum magnitude point of the first order differential. Using these ranges for the alpha threshold, and the specific value of 15%, have been found to accurately determine the alpha transition point such that accurate and useful features can be extracted based on the determined alpha transition point.

In some implementations of the method, the alpha threshold may be adjusted to reduce the influence of noise on the determining of the alpha transition point, and to improve the processing resource efficiency of the method. Therefore, determining the alpha transition point may further comprise: when no point between the maximum value of the first order differential of the breath waveform and maximum value of the breath waveform is less than the alpha threshold, increasing the alpha threshold. The alpha threshold may be increased by a predetermined amount. For example, the alpha threshold may be increased by 5% of the maximum magnitude point of the first order differential of the breath waveform.

After the alpha threshold has been increased, the values of the first order differential between (i.e., between points in time corresponding to) the maximum value of the first order differential and the maximum value of the breath waveform and/or the beta transition point can be compared with the increased alpha threshold in order to determine and define the alpha transition point. When no point meets this definition, the alpha threshold can be increased again and this process performed iteratively until the alpha transition point has been determined. The amount that the alpha threshold is increased may vary in different iterations.

In order to reduce unnecessary processing resources used during the method, an upper limit can be placed on the number of iterations performed (and therefore the number of times the alpha threshold is increased) and/or the value of the alpha threshold itself, and when one of these limits is reached the breath waveform is rejected.

The alpha transition point may also be determined using further alternative methods. One such alternative method of determining the alpha transition point comprises: calculating a line between the delta transition point and the maximum value of the breath waveform, or calculating a line between the delta transition point and the beta transition point; and, defining the alpha transition point based on the distance between the breath waveform and the calculated line.

For example, the alpha transition point may be defined as the point of the breath waveform, between the delta transition point and the maximum value of the breath waveform (or the beta transition point), that is furthest from the line calculated between the delta transition point and the maximum value of the breath waveform (or between the delta transition point and the beta transition point), where the distance between the breath waveform and the calculated line is measured using another line that is perpendicular to the (first) calculated line (between the delta transition point and the maximum value of the breath waveform or the beta transition point).

This may be implemented as an alternative method of determining the alpha transition point to that using the alpha threshold or used as a supplementary method of determining the alpha transition point to corroborate another method.

In an alternative implementation to the above algorithmic approach, determining the transition points may comprise: applying a trained machine learning model to a set of discrete samples of the breath waveform, the breath waveform representing a whole breath, wherein the machine learning model is configured to classify each sample into one of a plurality of output classes, each class representing a region of the breath waveform, and wherein the machine learning model is trained by: obtaining a label associated with each discrete sample of a plurality of breath waveforms, each breath waveform being represented by a set of samples representing a whole breath and each label indicating which of a plurality of output classes that sample corresponds to; and, training the machine learning model on the labels and the samples to learn to classify a sample of a set of samples representing a whole breath into a class of the plurality of output classes. This approach may provide consistency for feature engineering but may be difficult to consider all types of breaths and accuracy will scale with the volume of labelled input data.

Optionally, the method further comprises: extracting features from a plurality of breath waveforms recorded from the same user; determining a parameter of the extracted features; wherein the variability of the extracted features is also used to apply the trained machine learning model to classify the capnogram (and in some embodiments, to predict the cardiorespiratory disease), or wherein the variability of the extracted features is also used to train the machine learning model to create the classifying function (in some embodiments, the classifying function is a predictive function). The parameter may for example be variability of the extracted features over the time period the waveforms were recorded, or further the parameter may be a metric related to the features over time such as mean, median, standard deviation, a distribution or other suitable similarity score such as cosine similarity or distance. Variability of extracted features may be determined using two or more breath waveforms recorded from the same user. More preferably, the plurality of breath waveforms recorded from the same user comprises at least three breath waveforms recorded from the same user. It has been found that determining variability from at least three breath waveforms provides accurate classification and/or an accurate classifying function.

The time period used to determine the variability of extracted features may vary depending on the features being examined and/or the cardiorespiratory disease the machine learning model is predicting (or being trained to predict). The time period over which the breath waveforms were recorded may be, for example, multiple hours, multiple days, multiple weeks or multiple months. For some cardiorespiratory diseases, the variation in the extracted features over time is a particularly important indicator when predicting whether a capnogram was produced by a cardiorespiratory tract with that disease. For example, the time period may comprise two or more days, preferably the time period comprises five or more days, and more preferably the time period comprises ten or more days. For some cardiorespiratory diseases, such as obstructive airway diseases characterised by airway variability (e.g. asthma) the time period over the which the breath waveforms were recorded is less important, and accurate classification (or an accurate classifying function) can be achieved by determining variation without considering the time period.

Preferably, the plurality of breath waveforms are recorded at substantially regular intervals over the time period. For example, when the time period is five days, the plurality of breath waveforms includes a first breath waveform obtained from a first capnogram recorded on the first of the five days, a second breath waveform obtained from a second capnogram recorded on the second of the five days, a third breath waveform obtained from a third capnogram recorded on the third of the five days, and so on. More preferably, capnograms are recorded two or more times a day in order to assess variability of breath waveforms and extracted features across the day. For example, morning and evening variability has been found to be particularly effective for classification relating to asthma.

In certain examples, when predicting whether a capnogram was produced from a user with a cardiorespiratory disease, the plurality of capnograms should each be obtained from capnograms recorded by a single user.

A large variety of features of the breath waveform may be extracted using the determined transition points. For example, the extracted features may include at least one of: the time and/or CO₂ pressure at transition points or any other point of the breath waveform, the end-tidal CO₂, angles between linear fits either side of a transition point, duration of phases between transition points, the ratio of the angle between linear fits and the duration of the phase(s), the minimum, maximum, average, median and/or total CO₂ pressure during the phases between transition points, the respiratory rate, coefficients of lines fitted to the breath waveform using the transition point(s).

Further examples include: hyperbolic tangent fits, a delta angle, an alpha angle, a beta angle, a gamma angle, a delta angle start, a delta angle finish, an alpha angle start, an alpha angle finish, a beta angle start, a beta angle finish, a gamma angle start, a gamma angle finish and the time and CO₂ values at these points, the minimum CO₂ in the inspiratory baseline of the breath waveform, the minimum CO₂ in the expiratory upstroke of the breath waveform, the ratio of the alpha angle to the duration of the expiratory phase, the CO₂ at the centre of the gamma angle, the coefficients of a quadratic fit to the expiratory plateau, the time difference between any of the determined transition points, breath patterns and measures of disorder such as entropy.

Features relating to breath patterns are particularly useful for classifying capnograms relating to breathing pattern disorders. These can be detected by assessing periodicity of breath waveforms (e.g. using autocorrelation or frequency-domain identifiers) and comparing variability in breaths.

The delta angle start refers to the capnogram values at the delta angle start, for example the time and/or pCO₂ values at the delta angle start. This is the same for the start and finish points of the alpha, beta and gamma angles.

Extracting features of the breath waveform using the transition point(s) may comprise determining an angle of the transition point(s). The determination of an angle of the transition point(s) may comprise fitting a first linear function and a second linear function to the adjacent phases on either side of the transition point, and measuring the angle between the first and second linear functions. For example, if the angle of the alpha transition point is being determined then fitting the first/second linear function to the adjacent phases on either side of the alpha transition point means fitting the first linear function to the expiratory upstroke and the second linear function to the expiratory plateau.

Extracting features of the breath waveform using the transition point(s) may also comprise fitting a quadratic function to the expiratory plateau, and determining one or more coefficients of the quadratic function. This provides an indication of the “flatness” of the expiratory plateau which can be used to help determine characteristics of the respiratory tract associated with the breath waveform. Preferably, the quadratic function is fitted across the full length of the expiratory plateau from the alpha transition point to the beta transition point.

As a further example, extracting features of the breath waveform using the transition point(s) may comprise fitting a hyperbolic tangent function to the expiratory upstroke and/or the inspiratory downstroke, and determining one or more coefficients of the hyperbolic tangent function(s). These are coefficients such as the width and horizontal displacement parameters of the fitted hyperbolic tangent function(s). Preferably, the hyperbolic tangent functions are fitted across the full length of the expiratory upstroke (i.e., between the delta and alpha transition points) and the full length of the inspiratory downstroke (i.e., between the beta and gamma transition points).

Some machine learning or statistical inference models have been found to be particularly effective (e.g., high accuracy predictions) when used in the methods of the invention. Thus, it is preferred that the machine learning model comprises at least one of: logistic regression, a gradient boosting decision tree, a support-vector machine, ensemble methods such as AdaBoost, and a random forest. This is not an exhaustive list of models.

In some implementations of the first aspect, the trained machine learning model is further configured to output an indication of the importance of an extracted feature that led to the classification of the capnogram. That is, in some embodiments, the prediction of the cardiorespiratory disease. The method may include the step of outputting this indication. In this way, the indication may be used to evaluate the trained machine learning model, and to interpret the prediction by providing additional context for the prediction made.

According to a third aspect, there is provided a method for classifying one or more capnograms produced from a user, the method comprising: obtaining a plurality of breath waveforms from one or more capnograms produced from the user; normalising the duration of the plurality of breath waveforms to generate a plurality of normalised breath waveforms; generating an average breath waveform from the plurality of normalised breath waveforms (preferably using a generalised additive model, GAM); extracting features from the average breath waveform; and applying a trained machine learning model to the extracted features, wherein the trained machine learning model is configured to output a classification of the one or more capnograms from the features.

Similarly to the first aspect, the classification of the one or more capnograms may be used to predict a cardiorespiratory disease. These embodiments may be considered a method for predicting whether one or more capnograms were produced from a user with a cardiorespiratory disease, where the trained machine learning model is configured to predict a cardiorespiratory disease from the features.

That is, embodiments of the third aspect include a method for predicting whether one or more capnograms were produced from a user with a cardiorespiratory disease, the method comprising: obtaining a plurality of breath waveforms from one or more capnograms produced from the user; normalising the duration of the plurality of breath waveforms to generate a plurality of normalised breath waveforms; generating an average breath waveform from the plurality of normalised breath waveforms (preferably using a generalised additive model, GAM); extracting features from the average breath waveform; and applying a trained machine learning model to the extracted features, wherein the trained machine learning model is configured to predict a cardiorespiratory disease from the features.

In this way, a single, smooth, average breath waveform is generated from which features can then be extracted. The extraction of features from this average breath waveform can be carried out instead of or in addition to the extraction of features from individual breath waveforms, using the same techniques discussed above in implementations of the first and second aspects. Similarly, the determination of transition points and identification of artefacts may also be performed using the same methods discussed above in the first and second aspects. The skilled person will recognise the method steps relating to determining the transition points and artefacts are suitable for use in the third aspect.

Optionally, the duration and the amplitude of the plurality of breath waveforms are normalised to generate the plurality of normalised breath waveforms. That is, the normalised breath waveforms have been normalised with respect to duration and amplitude.

The third aspect of the invention may be combined with any of the first or second aspects to improve these aspects and is also separable from each of these aspects such that it can be implemented separately from these aspects.

According to a fourth aspect, there is provided a method for training a machine learning model to learn indicators of a capnogram, the method comprising: generating a plurality of average breath waveforms by, for each average breath waveform:
obtaining a plurality of breath waveforms from one or more capnograms produced from the user;
normalising the duration of the plurality of breath waveforms to generate a plurality of normalised breath waveforms; and
generating the average breath waveform from the plurality of normalised breath waveforms (preferably using a generalised additive model, GAM);
   extracting features from each of the plurality of average breath waveforms; obtaining a label for each average breath waveform indicating the capnogram classification of the corresponding one or more capnograms; and using the extracted features of the plurality of average breath waveforms and the corresponding labels, training a machine learning model to learn indicators of a capnogram to create a classifying function.

Similarly to the second aspect, in some embodiments of the fourth aspect the machine learning model is trained to learn indicators of a cardiorespiratory disease (i.e. indicators of a capnogram being produced from a user with a cardiorespiratory disease), with the capnogram classification of the corresponding one or more capnograms indicating whether the one or more capnograms were from a diseased respiratory tract and/or circulatory system.

That is, embodiments of the fourth aspect include a method for training a machine learning model to learn indicators of a cardiorespiratory disease, the method comprising: generating a plurality of average breath waveforms by, for each average breath waveform:
obtaining a plurality of breath waveforms from one or more capnograms produced from the user;
normalising the duration of the plurality of breath waveforms to generate a plurality of normalised breath waveforms; and generating the average breath waveform from the plurality of normalised breath waveforms (preferably using a generalised additive model, GAM);
   extracting features from each of the plurality of average breath waveforms; obtaining a label for each average breath waveform indicating whether the corresponding one or more capnograms were from a diseased respiratory tract and/or circulatory system; and using the extracted features of the plurality of average breath waveforms and the corresponding labels, training a machine learning model to learn indicators of a cardiorespiratory disease to create a predictive function.

Optionally, the duration and the amplitude of the plurality of breath waveforms are normalised to generate the plurality of normalised breath waveforms. That is, the normalised breath waveforms have been normalised with respect to duration and amplitude.

The fourth aspect of the invention may be combined with any of the first to third aspects to improve these aspects and is also separable from each of these aspects such that it can be implemented separately from these aspects.

Although several the techniques of the third and fourth aspects relating to generating an average waveform are described in the context of predicting whether one or more capnograms were produced from a user with a cardiorespiratory disease, the skilled person will recognise these average waveform techniques are not limited to use with cardiorespiratory diseases or predicting methods and can be implemented with any similarly shaped waveform.

Normalising the amplitude of the plurality of breath waveforms may comprise: extracting an end-tidal CO₂ value from each breath waveform; adjusting the amplitude of each of the plurality of breath waveforms such that each of the plurality of breath waveforms has the same end-tidal CO₂ value. The amplitude of the plurality of breath waveforms may be normalised based on various points of the individual breath waveforms such as the maximum value of the breath waveform or other transition points such as the alpha transition point. However, it has been found that normalising the waveforms based on the end-tidal CO₂ value consistently provides normalised waveforms which are well suited to use with the GAM to generate a smooth and accurate average breath waveform.

Extracting the end-tidal CO₂ value from each breath waveform may comprise, for each of the plurality of breath waveforms, determining: a beta transition point between the expiratory plateau and the inspiratory downstroke.

Preferably, obtaining the plurality of breath waveforms comprises: separating the one or more incoming capnograms into a plurality of capnogram sections, each representing a single breath waveform corresponding to a single respiratory cycle; and for each of said plurality of capnogram sections: determining a delta transition point between an expiratory baseline and an expiratory upstroke; determining a gamma transition point between an inspiratory downstroke and an inspiratory baseline; and extracting the portion of the capnogram section between the delta transition point and the gamma transition point to generate a breath waveform.

Generating an average breath waveform from the plurality of normalised breath waveforms may comprise: identifying whether any of the plurality of normalised breath waveforms are anomalous; and excluding said anomalous normalised breath waveforms when generating the average breath waveform (preferably by using a GAM). Excluding anomalous breath waveforms increases the usefulness of the resulting average breath waveform generated using the normalised breath waveforms. Anomalous breath waveforms may be identified without normalising the breath waveforms, however normalising the breath waveforms can improve the accuracy of any later anomaly detection.

Preferably, identifying whether any of the plurality of normalised breath waveforms are anomalous comprises: interpolating the normalised breath waveforms to all have the same number of data points; comparing the data points of each interpolated breath waveform to the data points from the other interpolated breath waveforms; and identifying any interpolated breath waveforms having one or more anomalous data points as being anomalous.

The interpolated breath waveform refers to the waveform resulting from the interpolation of the normalised breath waveform. This approach has been found to be less computationally demanding than other approaches to excluding anomalous breaths such as standard anomaly detection algorithms.

Preferably, the methods of the first aspect and the third aspect further comprise obtaining a capnogram from a user, wherein the capnogram comprises a breath waveform.

According to a fifth aspect, there is provided a method of diagnosing a cardiorespiratory disease, the method comprising the method according to any implementation of the first aspect or the third aspect, and further comprising: diagnosing the presence of a cardiorespiratory disease using the classification of the capnogram.

It will be apparent that the classification of the capnogram is used to diagnose the presence of the cardiorespiratory disease, and this diagnosing is performed away from the user who produced the capnogram corresponding to the classification.

According to a sixth aspect, there is provided an apparatus configured to perform the method of any the first aspect to the fifth aspect.

According to a seventh aspect, there is provided an apparatus for classifying a capnogram, the apparatus comprising: an obtaining module configured to obtain a breath waveform from a capnogram; a determining module configured to determine one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau; the apparatus further comprising an extracting module configured to extract features of the breath waveform using the transition points; and a prediction module configured to apply a trained machine learning model to the extracted features, wherein the trained machine learning model is configured to classify the capnogram from the features.

Embodiments of the seventh include an apparatus for predicting whether a capnogram was produced from a user with a cardiorespiratory disease, the apparatus comprising: an obtaining module configured to obtain a breath waveform from a capnogram; a determining module configured to determine one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau; the apparatus further comprising an extracting module configured to extract features of the breath waveform using the transition points; and a prediction module configured to apply a trained machine learning model to the extracted features, wherein the trained machine learning model is configured to predict a cardiorespiratory disease from the features.

According to an eighth aspect, there is provided an apparatus for training a machine learning model to learn indicators of a capnogram, the apparatus comprising: an obtaining module configured to obtain a breath waveform from each capnogram of a plurality of capnograms, and further configured to obtain a label for each capnogram indicating a classification of the capnogram; a determining module configured to determine one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau; an extraction module configured to extract features of the breath waveform using the one or more transition points; and a training module configured to use the extracted features of the plurality of breath waveforms and the corresponding labels of the respective capnograms to train a machine learning module to learn indicators of a capnogram to create a classifying function.

Embodiments of the eight aspect include an apparatus for training a machine learning model to learn indicators of a capnogram being produced from a user with a cardiorespiratory disease, the apparatus comprising: an obtaining module configured to obtain a breath waveform from each capnogram of a plurality of capnograms, and further configured to obtain a label for each capnogram indicating whether the capnogram was from a diseased respiratory tract and/or a circulatory system; a determining module configured to determine one or more transition points of the breath waveform, wherein the transition points comprise: a delta transition point between an expiratory baseline and an expiratory upstroke, a gamma transition point between an inspiratory downstroke and an inspiratory baseline, and an alpha transition point between the expiratory upstroke and an expiratory plateau; an extraction module configured to extract features of the breath waveform using the one or more transition points; and a training module configured to use the extracted features of the plurality of breath waveforms and the corresponding labels of the respective capnograms to train a machine learning module to learn indicators of a cardiorespiratory disease to create a predictive function.

According to a ninth aspect, there is provided an apparatus for classifying one or more capnograms produced from a user, the apparatus comprising: an obtaining module configured to obtain a plurality of breath waveforms from one or more capnograms produced from the user; a normalisation module configured to normalise the duration of the plurality of breath waveforms to generate a plurality of normalised breath waveforms; a generation module configured to generate an average breath waveform from the plurality of normalised breath waveforms (preferably using a generalised additive model, GAM); an extracting module configured to extract features from the average breath waveform; and a prediction module configured to apply a trained machine learning model to the extracted features, wherein the trained machine learning model is configured to output a classification of the one or more capnograms from the features.

Embodiments of the ninth aspect include an apparatus for predicting whether one or more capnograms were produced from a user with a cardiorespiratory disease, the apparatus comprising: an obtaining module configured to obtain a plurality of breath waveforms from one or more capnograms produced from the user; a normalisation module configured to normalise the duration of the plurality of breath waveforms to generate a plurality of normalised breath waveforms; a generation module configured to generate an average breath waveform from the plurality of normalised breath waveforms (preferably using a generalised additive model, GAM); an extracting module configured to extract features from the average breath waveform; and a prediction module configured to apply a trained machine learning model to the extracted features, wherein the trained machine learning model is configured to predict a cardiorespiratory disease from the features.

According to a tenth aspect, there is provided an apparatus for training a machine learning model to learn indicators of a capnogram, the apparatus comprising: an obtaining module configured to obtain a plurality of breath waveforms from one or more capnograms produced from the user; a normalisation module configured to normalise the duration of the plurality of breath waveforms to generate a plurality of normalised breath waveforms; a generation module configured to generate an average breath waveform from the plurality of normalised breath waveforms (preferably using a generalised additive model, GAM); an extracting module configured to extract features from each of the plurality of average breath waveforms; wherein the obtaining module is further configured to obtain a label for each average breath waveform indicating the capnogram classification of the corresponding one or more capnograms; and wherein the apparatus further comprises a training module configured to use the extracted features of the plurality of average breath waveforms and the corresponding labels to train a machine learning model to learn indicators of a capnogram to create a classifying function.

Embodiments of the tenth aspect include an apparatus for training a machine learning model to learn indicators of a cardiorespiratory disease, the apparatus comprising: an obtaining module configured to obtain a plurality of breath waveforms from one or more capnograms produced from the user; a normalisation module configured to normalise the duration of the plurality of breath waveforms to generate a plurality of normalised breath waveforms; a generation module configured to generate an average breath waveform from the plurality of normalised breath waveforms (preferably using a generalised additive model,

GAM); an extracting module configured to extract features from each of the plurality of average breath waveforms; wherein the obtaining module is further configured to obtain a label for each average breath waveform indicating whether the corresponding one or more capnograms were from a diseased respiratory tract and/or circulatory system; and wherein the apparatus further comprises a training module configured to use the extracted features of the plurality of average breath waveforms and the corresponding labels to train a machine learning model to learn indicators of a cardiorespiratory disease to create a predictive function.

In addition to the above examples, the classification of the capnogram may comprise a probability value corresponding to a severity of a respiratory disease. In other words, the output classification may comprise a probability value. Similarly, the classification of the capnogram may comprise a value indicative of a likelihood of the capnogram having a severity of a respiratory disease. The probability value may be indicative of a capnogram having a predetermined severity value, that is, a confidence that the capnogram is mild. The probability value may be indicative of whether a capnogram is likely a mild or severe capnogram and thus the probability value may represent where the capnogram falls on a range between mild and severe. That is, it is a continuous index that quantifies disease, e.g. COPD, severity with the model configured to use the continuous prediction probability outputs.

Further, the classifying function may be configured to predict a severity of a respiratory disease.

Additionally, the machine learning model may be trained based on capnograms labelled with a respective severity of a respiratory disease. Each capnogram or breath waveform may be labelled with a corresponding severity, either as a continuous variable or from a set of predetermined severities. Preferably each capnogram may be labelled as mild or severe, such that a probability value output by the model when implemented indicates a severity of the disease in a range.

According to further implementations, the machine learning model may be trained and configured to predict a likelihood that the capnogram is associated with a cardiorespiratory disease. Again we note that, by “disease”, we also mean subtype of a disease. The cardiorespiratory disease may be selected from a group comprising: COPD, Asthma, Asthma-COPD Overlap Syndrome (ACOS), small airways disease, chronic bronchitis subtype of COPD and emphysema subtype of COPD. It will be understood therefore that the diseases may be diseases and subtypes of disease but for clarity and brevity, we do not repeat the language “disease or subtype”.

The classification may be a binary classification or may be a multi-class classification. The machine learning model may be trained using labels belonging to (or indicating) a class representing that a capnogram is associated with a cardiorespiratory disease. The labels may further belong to a plurality of classes, each class representing that capnogram is associated with a cardiorespiratory disease, each class corresponding to a respective disease. The labels may further belong to a class representing that a capnogram is not associated with a cardiorespiratory disease. In this way, a binary classifier may be configured to predict a likelihood of disease or no disease, or a likelihood of one of a plurality of diseases. The problem may also be extended to multiclass classification to predict one of a plurality of diseases.

The machine learning model may be configured to output a likelihood that the capnogram is associated with the cardiorespiratory disease. The method may further comprise stratifying the output by comparing the likelihood to a set of threshold values, each strata representing a risk of the capnogram being associated with the cardiorespiratory disease. This improves clinical utility of the model, particularly when combined with an interpretable model.

According to an eleventh aspect, there is provided a computer readable medium comprising instructions which, when executed by a processor, cause the processor to: perform the method of the first aspect, the second aspect, the third aspect, the fourth aspect, and/or the fifth aspect described above.

Any claim which directly or indirectly depends on claim 1, which defines a method for classifying a capnogram, may also depend from claim 2, which defines a method for training a machine learning model to learn indicators of a capnogram.

### BRIEF DESCRIPTION OF DRAWINGS

Embodiments of the invention are described below, by way of example only, with reference to the accompanying drawings, in which:

FIG. 1A shows a breath waveform from a healthy patient;

FIG. 1B shows a breath waveform from a patient with a cardiorespiratory disease;

FIG. 1C shows a breath waveform including an artefact;

FIG. 2 shows an example capnogram;

FIG. 3 shows an idealised breath waveform;

FIG. 4 shows a method for predicting a cardiorespiratory disease according to examples of the present disclosure;

FIGS. 5A-5D show different breath waveforms together with the first order differential of the breath waveforms;

FIGS. 6A-6C show a schematic process flow for identifying hump artefacts, transition points, and features of the breath waveform;

FIG. 7 shows a method for training a machine learning model to learn indicators of a capnogram being produced from a user with a cardiorespiratory disease;

FIG. 8 shows a schematic of an example of the present disclosure applied to three components; and

FIGS. 9A to C illustrate the three components of FIG. 8 in schematic detail.

### DETAILED DESCRIPTION

Specific embodiments of the invention are discussed in detail below, relating to using a trained machine learning model to predict whether a capnogram was produced from a user with a cardiorespiratory disease, and training a machine learning model to learn indicators of a capnogram being produced from a user with a cardiorespiratory disease.

Recent hardware innovations, such as those described in WO2017174983 and WO201915800, the contents of which are each incorporated herein by reference, have led to it being possible to measure CO₂ concentration reliably and non-invasively at the mouth, closer to the airway and with a high time resolution. This has allowed for greater flexibility in pulmonary health assessment, providing more insight than would previously be recognised.

An objective of the present disclosure is to build a classifier that distinguishes between patients with and without a cardiorespiratory disease, such as a capnogram produced by lungs with COPD or Asthma, or a capnogram produced by a patient susceptible to heart failure. An objective is to provide explainability of objects so that clinicians are able to understand what led to the prediction and a computationally efficient process suitable to be deployed at the edge, for example so that an implementation stage can implement data processing, feature extraction and prediction using a trained model at the diagnosis site without cloud communication. Optionally, for example, the prediction methodology may be implemented on chip, i.e. on a handset, or tasks may be divided between the handset and a local computer.

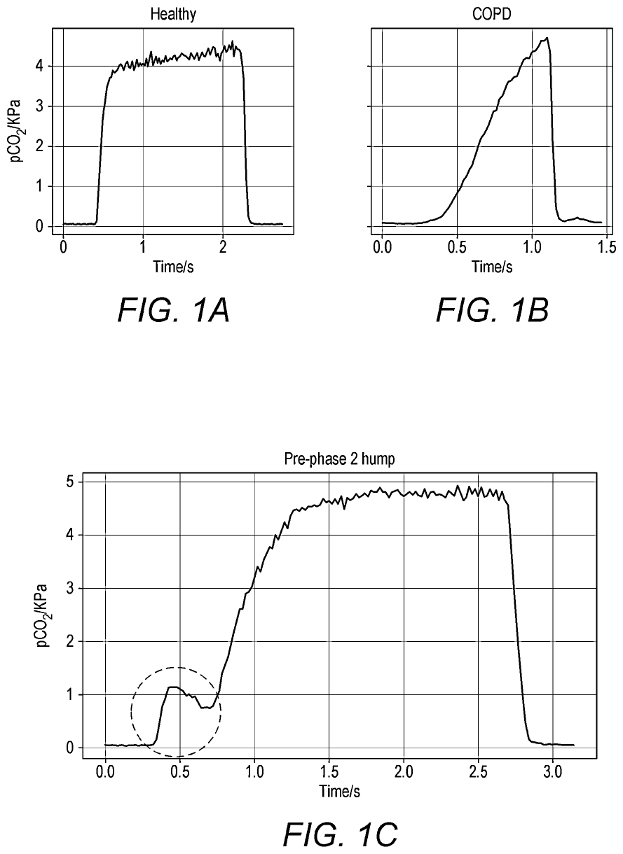

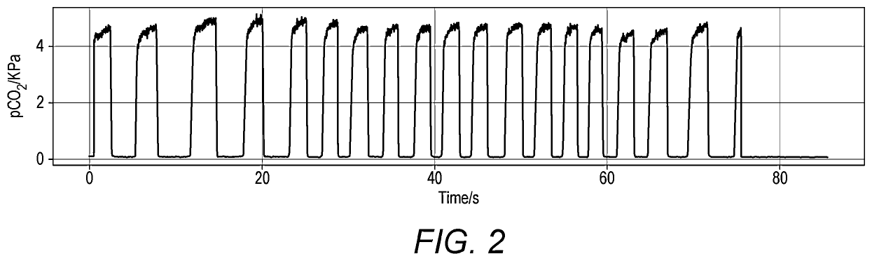

Previous academic work has made significant contributions to understanding how capnometry can be used to make diagnostic decisions. However, despite some impressive results with manual processing, healthcare providers are still awaiting a clinical implementation that has explainable, practical use. Methodologies proposed herein have particular beneficial use in the classification of patients with a variety of confounding diseases such as asthma, COPD, pulmonary fibrosis, CHF, motor neurone disease (MND), breathing pattern disorders, and pneumonia. FIGS. 1A-1C show three examples of different breath waveforms, where the breath waveforms plot the partial pressure of carbon dioxide (pCO₂) as a function of time as a patient performs a single breath by exhaling and then inhaling. The terms partial pressure of carbon dioxide, concentration of carbon dioxide, and carbon dioxide are used interchangeably throughout the description unless otherwise specified. FIG. 1A shows an example breath waveform produced by the breathing of a healthy patient, FIG. 1B shows an example breath waveform produced by the breathing of a patient with a cardiorespiratory disease (in this case COPD), and FIG. 1C shows an example breath waveform of a patient whose breathing produces a hump-like breath artefact in the waveform at the outset of the patient's exhalation.

Deoxygenated but CO₂ rich blood arrives through the pulmonary vasculature at the alveoli where gaseous exchange takes place. Perfusion exchanges O₂ in the ventilated air with carbon dioxide in the blood to facilitate respiration. The relaxation of the diaphragm then decreases the volume of the thoracic cavity during exhalation, thereby increasing pressure, and forcing CO₂ out through the bronchioles and into the environment. Although many implementations use volumetric capnometry, this disclosure focuses on time capnometry, which aims to measure the partial pressure of carbon dioxide (pCO₂) over time. It will be understood that aspects of the invention may also be applicable to volumetric capnometry. In an ideal gas mixture, the relationship of the total pressure to the individual gas partial pressures is given by

$${p}_{tot} = {\sum }_{i} {p}_{i}$$

where pᵢ is the partial pressure of gas i, which can be expressed as

$${p}_{i} = {p}_{tot} \frac{{n}_{i}}{{n}_{tot}}$$

with nᵢ as the number of molecules of gas i in any macroscopic volume and nₜₒₜ the number of molecules in the entire gas mixture in the same volume. At sea level, atmospheric pressure is pₐₜₘ=1 atm ˜101 KPa, with background pCO₂˜0.0417 KPa, dependent on the environment.

Typically, as a patient exhales, the concentration of CO₂ at the mouth increases sharply, from baseline, and typically reaches a plateau as diffusion begins to compensate. The inhalation then brings atmospheric CO₂ back towards the mouth, decreasing concentration back to baseline. The end of the plateau phase is referred to as the end-tidal CO₂ (ETCO₂), and is an important biomarker for anaesthetists.

Patients with an obstructive cardiorespiratory disease typically exhibit a more ‘shark-fin’ shape to their capnographic breath waveforms, as highlighted in FIG. 1B. During exhalation, damaged and/or inflamed alveoli and/or airways may limit perfusion, may degrade the elasticity of the alveoli walls, and may alter the physiology of the airways, altering the force at which gases can be exhaled, typically leading to a more gradual expiratory upstroke, a larger delta angle, and a steeper expiratory plateau.

It is clear from FIGS. 1A to 1C that the breath waveforms vary depending on the condition of a patient and so can be examined to predict the presence of a cardiorespiratory disease. The breath waveforms vary in a large variety of specific manners and so many different features may be extracted from a waveform and examined in order to predict the disease. These waveform variations and features will be discussed in more detail below.

As mentioned, recent innovations have led to the possibility of taking reliable recordings of pCO₂ directly from the mouth, with high sensitivity using a dedicated handset. It will be understood however that innovations disclosed herein are not limited to data derived from such a handset but description is provided for context. The patient performs normal tidal breathing through the mouthpiece for a period of ˜75 seconds resulting in a non-invasive and effort-independent experience.

Carbon dioxide strongly absorbs electromagnetic radiation of wavelength 4.3 μm or 15 μm, where the energy causes its intramolecular bonds to vibrate before being re-emitted at different wavelengths in different directions. An emitter in the handset emits photons of either of these wavelengths through the air pathway, where the unabsorbed photons are detected by a sensor whose output is dependent on the partial pressure of carbon dioxide within the air pathway's volume. The handset differs from conventional capnometers in that it does not have a reference channel; instead, the device self-calibrates on power up in reference to the background CO₂ level at the time of use. The sensor also has a fast response time because of a combination of the speed of respired breath through the sampling volume and because the sampling volume is adjacent to the mouth and so is unaffected by differential velocities due to wall drag when sampling distally from the mouth (as is the case with alternative technologies).

The resulting output is sampled at 10 kHz, and reported at 50 Hz, which is much higher than any other capnometry monitor on the market, whereafter the anonymous data is automatically, and securely uploaded via mobile network to a cloud platform. Here, it is run through a processing pipeline, as set out below in accordance with aspects of the disclosure, and made available for subsequent analysis. An example of a full raw breath record is displayed in FIG. 2.

FIG. 2 shows an example of a capnogram illustrating a tidal breath record, also plotting the partial pressure of carbon dioxide (pCO₂) as a function of time as a patient breathes. It will be apparent from comparing FIGS. 1A-1C with FIG. 2 that the capnogram of FIG. 2 illustrates over a dozen full breaths, along with a partial breath at the beginning and the end of the capnogram. The number of breath waveforms in a capnogram will vary depending on the length of time a patient's breathing is measured.

Input to the process which is the object of the present disclosure may accordingly be a set of digitised samples at a particular sampling rate, with each digital sample representing an amplitude of the pCO₂ at a particular point in time. The samples may be represented as a set of vectors, each with a corresponding time index. This time-series data represents the input to the methodology.

### Phase Segmentation

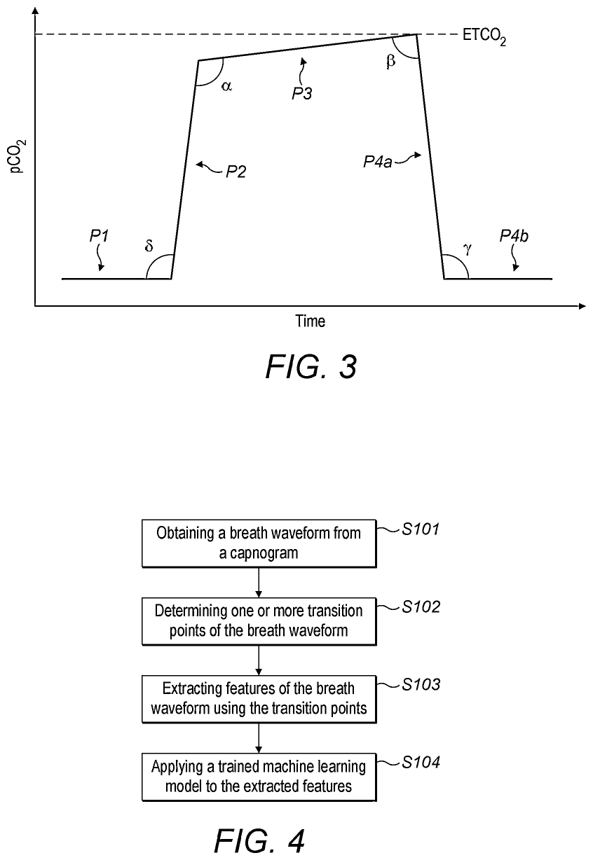

According to the present disclosure, a classifier has been developed that utilises several key geometric aspects identified common among breath waveforms which can be determined in order to facilitate feature extraction and the prediction of cardiorespiratory diseases from the respective biomarkers. These geometric aspects, or biomarkers, have hitherto not been capable of reliable and accurate identification by a computer, as will be explained in more detail below. FIG. 3 shows an idealised breath waveform with several of these geometric features highlighted.

The illustrated idealised waveform may be segmented into five linear segments or phases, from left to right as time progresses these are an expiratory baseline P1, an expiratory upstroke P2, an expiratory plateau P3, an inspiratory downstroke P4a, and an inspiratory baseline P4b. The expiratory baseline P1 is also referred to as phase 1, the expiratory upstroke P2 is also referred to as phase 2, the expiratory plateau P3 is also referred to as phase 3, the inspiratory downstroke P4a is also referred to as phase 4a, and the inspiratory baseline P4b is also referred to as phase 4b. Typically, as a patient exhales, the concentration of CO₂ at the mouth increases sharply, from the expiratory baseline P1, and reaches a plateau (the expiratory plateau P3) as diffusion begins to compensate for the increased CO₂ concentration. The inhalation then brings atmospheric CO₂ back towards the mouth of the patient, decreasing concentration back to baseline (the inspiratory baseline P4b). It will be apparent from the capnogram of FIG. 2 that the inspiratory baseline P4b of a first breath waveform may extend into or be the expiratory baseline P1 of a second breath waveform, and vice versa. The expiratory baseline P1 and inspiratory baseline P4b are so named in the context of a single breath cycle, i.e. a single waveform representing one breath cycle, to assist explaining individual breath waveform analysis.

As will be discussed below, determining the location of at least one of the transition points between these five segments allows for reliable and efficient feature extraction of the breath waveform but doing so is not straightforward. The delta transition point o is the point of the breath waveform bounding the expiratory baseline P1 and the expiratory upstroke P2. The alpha transition point α is the point of the breath waveform bounding the expiratory upstroke P2 and the expiratory plateau P3. The beta transition point β is the point of the breath waveform bounding the expiratory plateau P3 and the inspiratory downstroke P4a. The gamma transition point γ is the point of the breath waveform bounding the inspiratory downstroke P4a and the inspiratory baseline.

While the breath waveform in FIG. 3 is idealised with linear segments, this is not how breath waveforms appear in reality. Artefacts in a breath waveform such as the pre-expiratory upstroke P2 hump shown in FIG. 1C are common in capnograms and increase the difficulty of analysis (e.g. determining the location of transition point(s)) and feature extraction. Such artefacts are particularly problematic when automating the process or using a machine learning model to facilitate prediction. Many other types of artefacts are also found in breath waveforms and must also be accounted for to ensure that a prediction method is robust against as many types of artefacts as possible.

Noise such as that in the measured pCO₂ signal also makes it more difficult to accurately determine the transition points, as does the general shape of the waveform. For example, it will be appreciated that the alpha transition point α in FIG. 1A's “square wave” waveform is more clearly defined and so easier to determine accurately than the same alpha transition point α in FIG. 1B's “shark fin” waveform.

Each of these issues (e.g. artefacts and noise) may become more apparent in capnograms produced with higher measurement frequencies. However, if the issues are correctly accounted for when determining transition point(s) and extracting feature(s) from a breath waveform, then the extracted features are more accurate and so too are the resulting predictions.

Early implementations of computer-assisted methods consisted of a manual initial phase segmentation step that identifies each of the phases outlined in FIG. 3 through calculation of five features, including the maximum and minimum CO₂ values and the stroke gradients. A second step aimed to derive a template waveform by averaging the derived features between breaths before a third makes a comparison of subsequent breaths with this template. This enabled rejection of anomalous breaths with strange artefacts, the reason for which could be inferred by comparison of features to categories of breaths with known deformities. In similar approaches breaths were separated by pinpointing the positive and negative gradients, before a template waveform of CO₂ values was constructed from the average and outliers identified.

Rule-based methods have also been proposed, defining criteria on the absolute changes in CO₂ concentration as a way to extract phases for breath separation and further rules including the duration of breath were established for breath rejection. Other methods identified breath initiation and completion by finding inflection points in sliding windows where pCO2<16 KPa and rejected breaths based on pre-established extremes of physiological possibility. Another pre-processing approach includes fitting models to breaths. For example, segmenting breaths into phases by fitting a series of piecewise linear lines before splitting into individual breaths by application of logic to valleys. An analogous technique has been applied to identify segments by linearly modelling data in a sliding window of length 3 and rules applied to a plot of this slope fit over time.

Machine learning has also been used for breath segmentation where artificial neural networks (ANN) ingested the raw time series and output the start and ends of breaths. Similar solutions used an ANN on eight features derived from the waveform for poor-breath rejection.

These capnographs were mostly obtained from sedated patients, at low sampling rates, leading to greater uniformity in capnographic presentation.

None of these solutions provide an accurate, reliable technique for processing a high frequency noisy signal with artefacts leading to explainable and computationally efficient processing and classification.

FIG. 4 shows a method for predicting whether a capnogram was produced from a user with a cardiorespiratory disease according to examples of the present disclosure. Known methods are not able to accommodate the variety of artefacts which may influence the automated detection of the transition points.

In a first step, the method retrieves a single breath waveform from a capnogram (S101). This may be in the form of the digitised samples discussed above. Next, a set of transition points may be determined (S102). In examples, these are identified by analysing a first order differential of the waveform and maxima and minima of the signal. Preferably the first order differential is calculated using a time-based smoothing filter in conjunction with a first order derivative. Local minima and maxima of the signal may be caused by breathing artefacts or physiological artefacts and may interfere with the detection of the transition points. Output of this step may therefore be a set of start and end points of each of the phases, i.e. an index of the relevant sample for example, and optionally a first order differential and values at each of the identified points. From these elements, the method extracts a set of features (S103). As identified below, any number of features may be extracted from which the classifier is able to learn to predict a result from an unseen set of capnogram data. In an implementation phase, a trained machine learning model may be applied to the extracted features to predict presence of a disease in the user from which the capnogram was generated (S104).

It will be understood that in typical machine learning processes there may be three stages, training, validation and test. Each of these may be applied by different entities from features extracted from respective data sets using the above methodology.

In a general form, a supervised machine learning model takes input data, X∈R^{m×n} of m examples and n features sampled from χ, alongside paired output data, Y∈R^{m×k} sampled from Υ, as an m×k matrix of the corresponding m examples and k outputs. The objective is to use an algorithm, A, that uses this data to create a function, f∈γ^{χ}, that can take any unseen x∈X and output a prediction y∈Υ:

A: (X×Υ)→(f: X→Υ).

In this problem statement, X is the set of featurised capnograms, and Υ={Not COPD, COPD} :={0,1} are the labels that specify whether the capnogram was from non-COPD or COPD diseased lungs. A distinction is made between healthy and non-COPD, as patients without COPD could have other diseases that affect the capnography waveform.

In step S101, a breath waveform is obtained from a capnogram. When the capnogram includes a plurality of breaths (these may be full breaths and partial breaths) as in the example of FIG. 2, then obtaining a breath waveform includes splitting the capnogram into sections. When splitting the capnogram into sections, each capnogram section should preferably consist of a single breath waveform corresponding to a single respiratory cycle (i.e. a full breath or a partial breath if sampling of capnography data was started or stopped mid-breath).

One of the particular benefits of the present methodology is the ability to perform prediction accurately and reliably based on a single waveform. Preferably pre-processing steps such as de-noising and breath separation may take place but these are not essential and not described. A further pre-processing step to form a stylised single waveform is a further example of the disclosure and described below.

In step S102, one or more transition points of the waveform are determined. In particular, the determined transition points include one or more of the delta transition point δ, the gamma transition point γ, and the alpha transition point α.

These transition points may be determined through a number of methods. Due to its high accuracy and compatibility with automation and machine learning, a preferred method includes determining a first order differential of the breath waveform. It has been found that the first order differential of the breath waveform may be used as a reference against the breath waveform itself to determine several of the transition points.

Determining the first order differential of the waveform also enables the breath to be screened for viability to save computing resources. For example, the first order differential can be compared to a differential template and, when the first order differential of the breath waveform is not consistent with that template, the breath waveform will be rejected and analysis of the waveform will cease. The nature of the template and level of inconsistency permitted before the rejection threshold can vary depending on the details of the waveform analysis; for example, which transition points are to be identified and which features of the breath waveform are intended to be extracted.

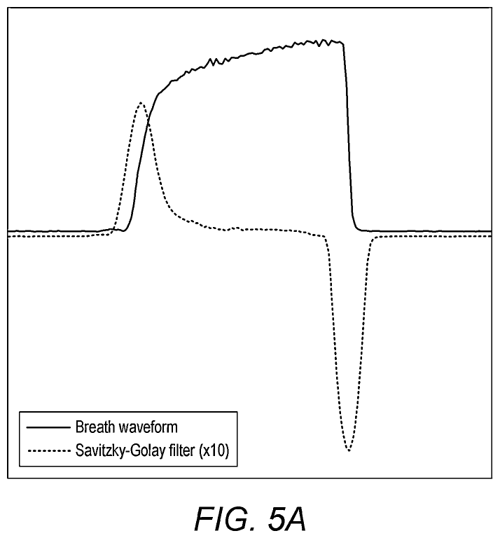

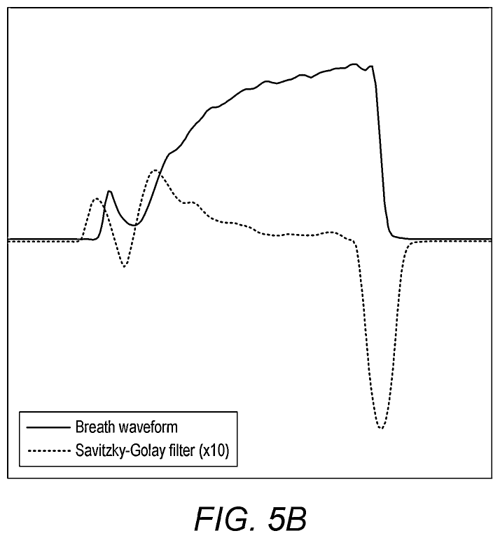

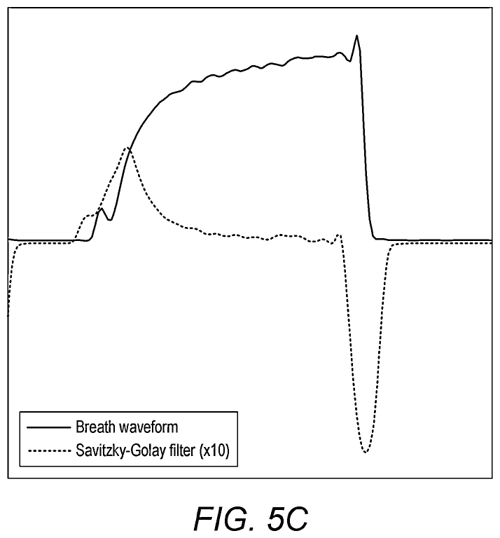

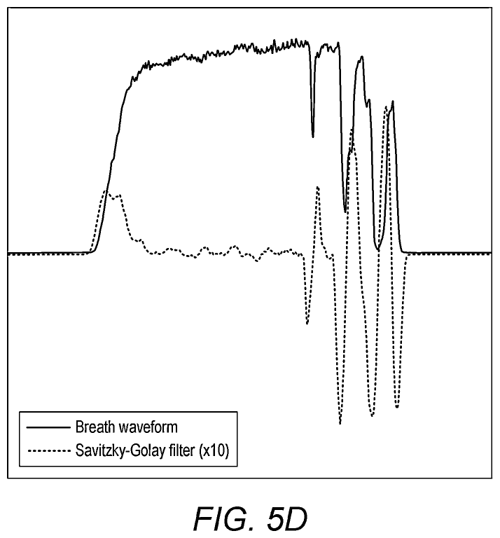

FIGS. 5A to 5D show several examples of breath waveforms overlayed by their first order differential, where the first order differential was determined in conjunction with a Savitsky-Golay (SG) filter applied to the breath waveform. The scales of the SG filter first order differential and breath waveform have been altered in these figures to facilitate easier comparison of the SG filter and the breath waveform. As will be apparent from FIGS. 5A to 5D, and in particular FIG. 5A when compared with the idealised waveform of FIG. 3, the peaks of the first order differential may substantially correspond, or be otherwise adjacent to, the transition points of the breath waveform and so may be used as part of determining the transition points.

It will be apparent from FIGS. 5B, 5C, and 5D that artefacts in the breath waveform have a significant effect on the shape of the waveform and the first order differential. For example, the pre-expiratory upstroke P2 hump artefact shown in FIGS. 1C, 5B and 5C. These artefacts should preferably be identified and accounted for during waveform analysis (e.g., during determining of transition points and/or feature extraction). This is particularly beneficial when automating the step of determining the transition points.

Preferably, when a hump artefact is identified in a breath waveform it is accounted for during further analysis of the breath waveform. For example, if a hump artefact produces the maximum magnitude points on the first order differential, rather than this being caused by the expiratory upstroke P2 or the inspiratory downstroke P4a, the first order differential can account for the hump artefact by being normalised with respect to a peak caused by the upstroke P2 or the downstroke P4a instead of the hump artefact. Various thresholds used in determining transition point(s) (discussed below) may then be defined based on the re-normalised first order differential to avoid influence from the hump artefact. Alternatively, the hump artefact may be accounted for by ignoring the artefact, for example by ignoring or removing the data associated with the hump artefact (in effect, subtracting the hump out), or adjusting the weighting applied to the data associated with the hump artefact when determining the one or more transition points.

Artefact identification may be performed through several methods. Due to its high accuracy and compatibility with automation and machine learning, a preferred method includes performing peak detection operations on the breath waveform, and optionally also determining and/or using the first order differential of the breath waveform.

As part of the artefact identification, peak detection is performed to identify local minima of the breath waveform. Prominent minima may then be identified from the local minima. Identifying the prominent minima is particularly useful when the breath waveform is a noisy signal. When one or more prominent minima are found, the local area of the waveform is examined to determine whether there is a hump artefact and, when present, identify the hump artefact. For example, it will be apparent from FIG. 1C that there is a prominent local minima before the expiratory upstroke P2 and after a hump artefact, searching for local maxima near this prominent minimum will identify the pre-expiratory upstroke P2 hump artefact.

When no prominent minima are identified (e.g., due to a noisy breath waveform), the first order differential of the breath waveform may be used to search for hump artefacts. As shown from comparison of FIG. 5A with FIGS. 5B and 5C, the first order differential (in this case, an S-G filter) is significantly affected by the presence of a pre-expiratory upstroke P2 hump artefact, i.e. during the expiratory baseline P1, and so can be used to identify hump artefacts that would otherwise have been missed. For example, in FIG. 5C the S-G filter increases and then plateaus for a short time at an inflection point before increasing again, indicating the smaller (relative to that of FIG. 5B) hump artefact in the breath waveform.

When a hump artefact is identified, either with or without the use of a first order differential, it is accounted for during the determining of the transition point(s) to ensure accurate definitions of the transition point(s). For example, after identifying a hump artefact the first order differential of the breath waveform may be normalised or re-normalised based on the maximum magnitude minima/maxima of the first order differential other than the hump artefact.

To avoid unnecessary use of computing resources, the region of the breath waveform considered during these steps (e.g. the peak detection and/or prominent minima identification) can be limited. For example, the maximum value of the breath waveform is identified and the breath waveform is divided in time into a first section and a second section. The first section is a period of the breath waveform not including the maximum value of the breath waveform, and the second section is a period of the breath waveform that includes the maximum value of the breath waveform. To save computing resources and avoid searching for a hump artefact during the inspiratory phases (P4a and P4b), the steps for identifying the hump artefact (e.g. identifying local minima, or searching for hump artefacts when at least one prominent minima is identified) are performed in the first section of the breath waveform and not in the second section of the breath waveform.

It has been found that minima corresponding to hump artefacts in capnograms typically have pCO₂ values below 2 kPa. In view of this, a predetermined threshold (herein the hump artefact threshold) may be implemented to further reduce the processing resources required when searching for a hump artefact. This may be implemented in a variety of manners, for example by discarding any minima detected where the minima is above the hump artefact threshold, or by not searching for these minima above the hump artefact threshold in the first instance. The hump artefact threshold may be used instead of the first section and second section divide described above, or in combination with that sectional divide of the breath waveform.

Even if no prominent minima are identified there may still be a hump artefact(s) present in the breath waveform. For example, this could be the case in FIG. 5C depending on the peak detection method used or the techniques used to identify prominent minima. When no prominent minima are identified, the first order differential of the waveform may be used to identify whether or not the breath waveform contains a hump artefact. The first order differential is reviewed for an inflection point region where the differential may be compared to a predetermined threshold.

Preferably, in order to reduce processing resources used, the first order differential is only reviewed in the region between the start of the first order differential and the maximum value of the first order differential.

Alternatively, rather than identifying local minima of the breath waveform and identifying prominent minima from the local minima, the hump artefact(s) can be identified using local maxima of the breath waveform (and corresponding prominent maxima).

When hump artefact(s) are present, the steps of identifying and accounting for any artefact(s) should be performed before identifying the transition point(s) for the most accurate definition of the transition point(s).

The delta transition point δ may be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the delta threshold). For example, the delta threshold may be a value equal to 10% of the maximum magnitude point of the first order differential. This delta threshold (and other thresholds) may also be found by normalising the first order differential up to 1 with respect to the maximum magnitude point of the first order differential (after accounting for any hump artefact(s). The location of the delta transition point δ in the time dimension may then be defined as the first point in time at which the first order differential of the breath waveform is above the delta threshold (after accounting for an identified hump artefact). That is, the breath waveform value corresponding to that point in time is the delta transition point δ between the expiratory baseline P1 and the expiratory upstroke P2.

The gamma transition point γ may also be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the gamma threshold). For example, the gamma threshold may be a value equal to the negative of 5% of the maximum magnitude point of the first order differential. The method also comprises determining the minimum value of the first order differential. The location of the gamma transition point γ in the time dimension may then be defined as the first point in time, after the minimum value of the first order differential of the waveform, at which the first order differential is higher than the gamma threshold. That is, the breath waveform value corresponding to that point in time is the gamma transition point γ between the inspiratory downstroke P4a and the inspiratory baseline P4b.

The alpha transition point α may also be determined using the first order differential of the breath waveform. A preferred method comprises determining the maximum magnitude point of the first order differential (i.e. the magnitude of the highest peak or lowest trough of the first order differential) and using this to define a predetermined threshold (herein referred to as the alpha threshold). For example, the alpha threshold may be a value equal to 15% of the maximum magnitude point of the first order differential. The method also comprises determining the maximum value of the first order differential and the maximum value of the breath waveform. In some examples the maximum magnitude point of the first order differential may also be the maximum value of the breath waveform, though this is not always the case and will depend on the specific breath waveform being analysed. The location of the alpha transition point α in the time dimension may then be defined as the first point in time after the maximum value of the first order differential at which the first order differential is less than the alpha threshold. That is, the breath waveform value corresponding to that point in time is the alpha transition point α between the expiratory upstroke P2 and the expiratory plateau P3.

In some examples of the method, the alpha threshold may be adjusted. For example, when no point of the first order differential, between the maximum value of the first order differential and the maximum value of the breath waveform, is less than the alpha threshold, the alpha threshold may be increased and the values of the first order differential (between the same boundaries) can be compared against the increased alpha threshold. This process can be performed iteratively until the alpha transition point α is defined, or until an upper limit of the number of iterations performed is reached. If this upper limit is reached the breath waveform may be rejected or the analysis of the breath waveform may continue.

To save processing resources, the location of the alpha transition point α in the time dimension may alternatively be defined as the first point in time between the maximum value of the first order differential of the breath waveform and the maximum value of the breath waveform (or the beta transition point β), at which the first order differential is less than the alpha threshold. The alpha transition point α is known to be located in the breath waveform before the beta transition point β and very likely to be before the maximum value of the breath waveform, so these limitations prevent unnecessary data analysis when determining the alpha transition point α.

An alternative method for determining the alpha transition point α comprises calculating a linear line between the delta transition point δ and the maximum value of the breath waveform (or the beta transition point), and defining the alpha transition point α based on the distance between the breath waveform and the calculated line. For example, the alpha transition point α may be defined as the point of the breath waveform, between the delta transition point δ and the maximum value of the breath waveform, that is furthest from the calculated line, when the distance between the breath waveform and the line is measured using a second linear line that is perpendicular to the calculated line.

Determining the one or more transition points may also include determining the beta transition point β between the expiratory plateau P3 and the inspiratory downstroke P4a. The beta transition point β may be used to extract additional features from the breath waveform and to help determine or verify other aspects of the waveform such as the alpha transition point α and a hump artefact.

Peak detection operations may be used to determine the beta transition point β. The peak detection operations are performed to identify local maxima of the breath waveform. Prominent maxima may then be identified from the local maxima; this is particularly useful when the breath waveform is a noisy signal. Prominent maxima may be identified by comparing each maximum identified to a threshold or by comparison amongst the set of maxima. Optionally, as it is expected that the CO₂ value of the beta transition point β will be above a predetermined threshold (herein a maxima threshold) for viable breath waveforms, CO₂ values below the maxima threshold may be ignored (e.g. by removing these values or setting them equal to 0 during the peak detection) to reduce noise from values below the maxima threshold during the peak detection.

When no prominent maxima are identified, this typically indicates the breath waveform is a bad sample and so the waveform is rejected without further unnecessary processing being performed. When only a single prominent maximum is identified, the location of the beta transition point β in the time dimension may then be defined as the time point of the single prominent maximum. When a plurality of prominent maxima are identified, the most prominent maximum is determined and this is taken as the beta transition point β. A variety of methods may be used to determine the most prominent maxima; one method uses a two-step prominence-based algorithm which compares the height of each maximum to the surrounding area and, if two or more maxima are not filtered out at that stage, compares the heights of the remaining maxima. If the most prominent maximum cannot be determined, for example in a reasonable number of iterations, then the first of these prominent maxima is taken as the beta transition point β. If other transition points are known, then these points may be used to assist determining the beta transition point β. For example, the beta transition point β is before the gamma transition point γ so any anomalous prominent maxima after the gamma transition point γ will not be considered during determining of the beta transition point β.

When the beta transition point β is known, this point may be used in the above-described method in place of the maximum value of the breath waveform. That is, the location of the alpha transition point α in the time dimension may then be defined as the first point in time, between the maximum value of the first order differential of the breath waveform and the beta transition point β, at which the first order differential is less than the alpha threshold.

The beta transition point β may also be used in the alternative method for determining the alpha transition point α described above. Instead of calculating a linear line between the delta transition point δ and the maximum value of the breath waveform, the linear line is calculated between the delta transition point δ and the beta transition point β. The alpha transition point α may then be determined based on this calculated line in the same manner described above.

Similarly, when identifying a hump artefact using the method described above, the breath waveform may be divided in time into a first section not including the beta transition point β and a second section including the beta transition point β. As previously described, the steps for identifying the hump artefact (e.g., identifying local minima, or searching for hump artefacts when at least one prominent minimum is identified) are performed in the first section of the breath waveform and not in the second section of the breath waveform.

Above we have described how a first-order differential (or a higher-order differential or a combination thereof) could be used to identify a transition point and compensate for noise and artefacts algorithmically. An alternative approach could be to analyse the point-by-point difference in the samples. For example, the method would take the point-by-point difference in the series, i.e. if the series is {t₀, t₁, t₂, . . . , tₙ}, this would yield a time series of {t₁-t₀, t₂-t₁, . . . , tₙ-tₙ₋₁} that is one shorter than the original. t₀ would be added to the beginning so the final series is {t₀, t₁-t₀, t₂-t₁, . . . , tₙ-tₙ₋₁}.

That is, the difference between each value or sample represents the change in the curve of the capnogram. This can be analysed to identify the transition points, factoring in noise and artefacts in a similar manner to that above. In summary, in addition to the approach described of having a time-based smoothing filter and first-order differential, a higher-order differential or a point-by-point difference in the time-series capnogram data (i.e. the values of the curve) could also be used to identify the shape of the curve and the transition points between phases.

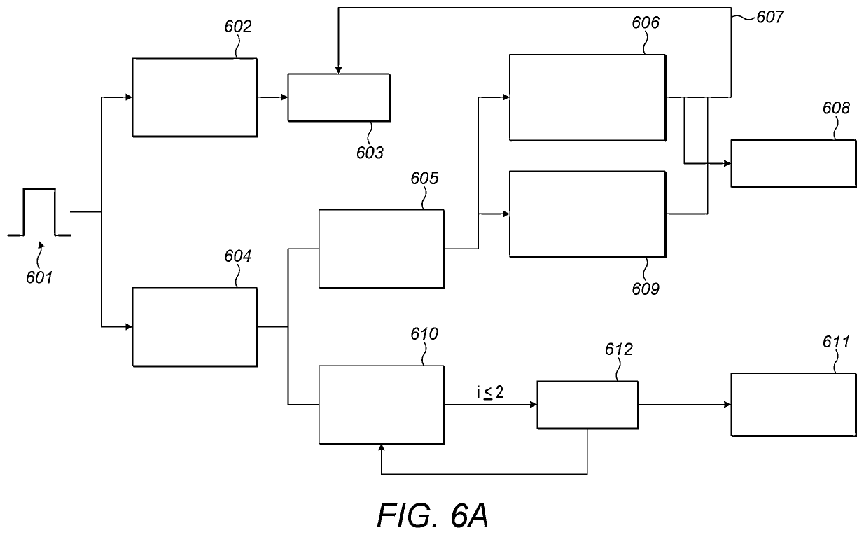

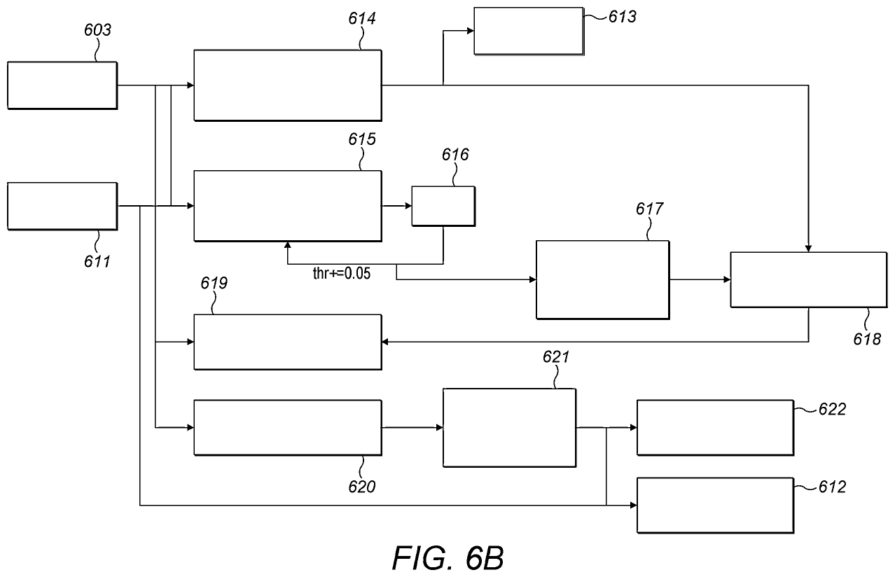

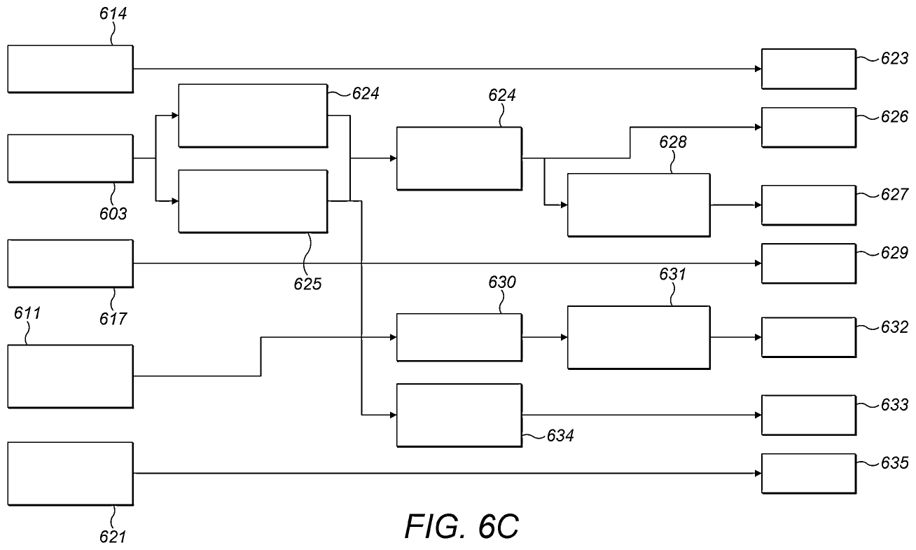

FIGS. 6A, 6B and 6C illustrate these above steps in the form of a process flow.

In a first step illustrated in FIG. 6A, the inhalation and exhalation phases are extracted by identifying the start location of the inhalation phase. The input to the process is the digitised samples of capnogram data 601. A first order Savitzky-Golay Differential is calculated using an appropriate Savitzky-Golay filter 602 and normalised 603. If this is not consistent with a good breath, it may be rejected for example by comparison with a template shape of differential or through threshold comparisons or other suitable means to identify the differential is expected.

Next, from the sampled data 601, a peak detection algorithm finds local maxima and minima 604. From a subset of the minima, if no prominent minima are detected, e.g. identified using a bidirectional local window 605, the differential is analysed 606 to identify additional humps prior to exhalation start. If these are present 608 the differential may be re-normalised 603 and the process begun again. The differential may identify no humps are present in the sampled data. If multiple minima are found in the subset, the samples are checked for a minimum in the first half of the breath, i.e. the cycle, where the minimum is less than a threshold (i.e. the hump artefact threshold). The threshold may preferably be selected as less than 2 kPA which is selected so as to identify the hump is unlikely to be on the plateau.

From the local maxima identified by the peak detection algorithm, it may be identified that the start of the inhalation is at the end of the breath. Such edge cases can be suitably processed. From a subset of the most prominent maxima 610, e.g. identified by a unidirectional forward window, the location of the start of the inhalation may be identified if only one maximum is detected 611. The waveform may be rejected if there are no maxima or if the resulting phase is too short. If there is more than one prominent maximum identified 612, the iteration described above may be performed to identify the start of the inhalation by comparing each maximum to a threshold or to each other, or alternatively taking the first maximum as the start of the inhalation phase.

In a second step, illustrated in FIG. 6B, from the normalised differential, i.e. the S-G filter differential, and the location in the waveform defined as the start of the inhalation, the method may identify the transition points and the start and end of each phase of the breath.

The location of the end of phase 1 613, may be identified as the first location in which the differential exceeds a positive threshold 614. Preferably the threshold may be 0.1 when the differential has been normalised up to 1 with respect to the maximum magnitude point of the differential (after accounting to any hump artefact(s)). The threshold is positively selected through detailed research and analysis. If the differential does not exceed the threshold, the waveform may be rejected.

From the differential, the value of the differential between the inhalation start location and the maximum point of the differential may be compared to a threshold 615. If there are too many points identified, i.e. above 20 locations, the waveform may be rejected. The threshold may be iteratively incremented 616, e.g. by 0.05, until fewer points are identified. The location 617 of the end of phase 2 can then be identified from this comparison 615. From the two identified locations 613, 617, the duration of phase 2 can be identified 618.

The end of phase 3 may be identified from the previously identified inhalation start 611. Therefore, the duration of phase 3 619 may be identified by comparing the end of phase 2 617 and the inhalation start 611.

From the differential, the first point to exceed a negative threshold between the minima of the differential and the end of the differential may be identified 620. The location of this point and the location of the minimum of the differential may thus be used to identify the end of phase 4a 621. The end of phase 4b 622 may be defined as the range of the end of phase 4a and the end of the waveform.

The duration of phase 4a 612 may be the range of the location defined as the start of the inhalation phase 611 above, i.e. the end of the phase 3, and the end of phase 4a 621.

Accordingly, the output of these two illustrated processes in FIGS. 6A and 6B, may be a normalised first order differential 603, the inhalation start location 611 and the start and end points of each phase of the waveform, i.e. the duration or ranges of the phases 613, 618, 619, 612, 622.

In a further optional example of the present disclosure, the segmentation may be further separated into angular phases. An example of such a process flow is illustrated in FIG. 6C.

The start of the delta angle 623 is defined as the end of phase 1 614. The first significant maximum 624 of the differential 603 after the local minimum 625 but before the last point in the differential attains its minimum 626 is defined as the end of the delta angle 626. The local minimum 625 is used to avoid any humps at the outset of the waveform.

The start of the alpha angle 627 is defined as the first time after the first significant maximum 624 that the differential goes below a threshold 628, preferably selected in the range from 0.2 to 0.9, and more preferably as 0.5. The end of the alpha angle 629 is defined as the end of phase 2.

The start of the beta angle is defined as the inhalation start location. The end of the beta angle 632 is identified as the first time after the inhalation start that the differential is lower than a negative threshold 631, preferably selected in the range from −0.4 to −1, and more preferably as −0.9.

The start of the gamma angle is defined as the first time after the minimum 625 of the differential that the differential exceeds a negative threshold 634, preferably selected in the range from −0.6 to −1, and more preferably as −0.9. The end of the gamma angle may be defined as the end of the phase 4a 621.

These angular features may be optionally used to identify further beneficial features, as discussed below, notably preferably based on the transition points.

In step S103, features of the breath waveform are extracted using the determined transition points.

Deriving important features (i.e. biomarkers) from a capnogram allows a machine learning model to be trained and configured to make predictions based on these features. Many different features of the breath waveform may be extracted using a variety of methods; a non-exhaustive list of examples is provided below. It should be noted that the benefits of each of these identified features may be separable from the overall method in which we present their context.

Extraction of biomarkers, or features, from capnography has been a significant point of focus in academia. These presented features are each particularly beneficial and may not hitherto have been described or calculated automatically. In any case, it has not been previously described that such features are extracted based on the transition points, nor how to effectively calculate them without manual intervention.

In a first set of features, the angle at a transition point may be determined by applying a linear fit to each phase on either side of the transition point and calculating the angle between them. For example, an alpha angle (the angle at the alpha transition point α) may be determined by fitting linear lines to the expiratory upstroke P2 and the expiratory plateau P3 and calculating the angle between them at the alpha transition point α where the fitted lines intersect. The linear lines may be fitted between two transition points (e.g., a linear line fitted to the expiratory upstroke P2 between the delta transition point δ and the alpha transition point α), or between the subject transition point (in this case the alpha transition point α) and another point along the adjacent phase (e.g., in this case a point along the expiratory upstroke P2 or the expiratory plateau P3). The nature of the linear fit may depend on the shape of the breath waveform. It has been found that different features are indicative of different physical characteristics. For example, in the context of the features and transition points as defined in this document, the alpha angle is indicative of obstructive airways disease.

As described above in relation to FIG. 6C, angle start and finish values (e.g., the time value and CO₂ value) are also important features that have been discovered to consistently drive the machine learning models effectively and improve their performance, and lead to further features which also improve the machine learning models. Further examples of these features are described below.

The delta angle start may be defined as the delta transition point δ (i.e., the end of the expiratory baseline P1) while the delta angle finish requires further calculation to determine. The first order differential of the breath waveform may be used to determine the delta angle finish. The location of the delta angle finish in the time dimension may then be defined as the point in time (from the start of the breath waveform or, when identified, after a hump artefact) corresponding to the first significant maximum of the first order differential of the breath waveform. Alternatively, the delta angle finish may be defined as the first significant maximum (i.e. relative to a predetermined threshold) of the differential after the local minimum before the location of the minima of the differential.

The location of the alpha angle start in the time dimension may be defined as the first point in time, after the first significant maximum of the first order differential (i.e., the first point after the delta angle finish), at which the first order differential of the breath waveform is below a predetermined threshold (herein referred to as the alpha start threshold). For example, the alpha start threshold may be a value from 20% to 90% of the maximum magnitude point of the first order differential, and more preferably is 50% of the maximum magnitude point of the first order differential. The alpha angle finish may be defined as the alpha transition point a (i.e., the end of the expiratory upstroke P2).

The beta angle start may be defined as the beta transition point β (i.e., the start of the inspiratory downstroke P4a). The location of the beta angle finish in the time dimension may be defined as the first point in time after the beta angle start that the first order differential of the breath waveform is below a predetermined threshold (herein referred to as the beta finish threshold). For example, the beta finish threshold may be a value from 40% to 100% of the negative of the maximum magnitude point of the first order differential of the breath waveform, and more preferably is 90% of the negative of the maximum magnitude point of the first order differential of the breath waveform.

The location of the gamma angle start in the time dimension may be defined as the first point in time, after the minimum value of the first order differential of the breath waveform, at which the first order differential is higher than a predetermined threshold (herein referred to as the gamma start threshold). For example, the gamma start threshold may be a value from 60% of the negative of the maximum magnitude point of the first order differential of the breath waveform, and more preferably is 90% of the negative of the maximum magnitude point of the first order differential of the breath waveform. The gamma angle finish may be defined as the gamma transition point γ (i.e., the end of the inspiratory downstroke P4a).

In specific implementations, as there is often a subtly varying diverse number of breaths per capnogram and breath shape within a capnogram, a median may be calculated for the per-breath features in each capnogram. Example features in implementations include the following: α, β, γ, and δ angles; gradients and residuals derived from fitting curves to phases, such as the expiratory plateau; absolute and short-term variability of pCO2; curvature and other higher-order time-based features such as the ratio of the expiratory to inspiratory phase; second order area ratios in quadrants of the expiratory phase and area under the curve (AUC), such as those calculated in volumetric capnography.

Features associated with the alpha region of the capnogram waveform have been found to be the most important drivers of learning. The identified features contributing most to a model's decision have been found from the a angle region, which characterizes the rate at which gas from the upper airways (CO2-poor) gives way to mixed alveolar gas from the lower airways (CO2-rich).

In step S104, a trained machine learning model is applied to the extracted features. The trained machine learning model is configured to predict the presence of a cardiorespiratory disease from the features. In particular, the trained machine learning model is configured to predict whether a capnogram (from which the features were extracted) was produced from a user with a cardiorespiratory disease.

Examples of applicable machine learning algorithms include logistic regression, a gradient boosted decision trees, a support-vector machine, AdaBoost, and a random forest, though it will be appreciated other algorithms are also suitable. Some machine learning models, such as the logistic regression model, allow for the influence of each extracted feature on the prediction to be examined. Some models, such as the gradient boosted decision tree model, maintain a high level of interpretation while being resilient to overfitting, helping to ensure the model is generalisable to new data. It will be appreciated that the invention can be implemented using a variety of machine learning models other than those listed in the description.

The trained machine learning model may be configured to provide additional information as part of the prediction or together with the prediction. For example, the trained machine learning model may predict the type of cardiorespiratory disease the user had when they produced the capnogram, or the severity or subtype of cardiorespiratory disease. The model may also provide a level of certainty associated with the prediction and/or highlight which extracted features led to the prediction outcome and to what extent the (different) feature(s) contributed.

As part of step S104, the trained machine learning model may be applied to features extracted from a single breath waveform or from a plurality of breath waveforms. The plurality of breath waveforms may be from a single capnogram (i.e. a series of breath waveforms recorded by a user in a single period of breathing) or from a plurality of capnograms taken at different times. When features are extracted from a plurality of capnograms, these capnograms may be obtained (i.e. recorded by the user) over an extended period of time. For example, capnograms may be repeatedly recorded by a user several times in a day for a period of a plurality days, weeks or months. Extracting features from multiple breath waveforms obtained over an extended time period (i.e. multiple capnograms as opposed to from a breath waveform(s) from a single capnogram) allows the variability of the extracted features to be determined and provide a more accurate prediction of whether the user had a cardiorespiratory disease when they produced a capnogram. For example, it has been found that the cardiorespiratory disease Asthma may be more accurately predicted when the variability of extracted features is assessed using at least three capnogram recordings, and preferably where the recordings were made over a period of at least five days, and more preferably where the recordings were made over a period of at least ten days. In comparison, the variability of extracted features may be less important for predicting the presence of the cardiorespiratory disease COPD and so this can be predicted very accurately using the disclosed methods on a single breath waveform.

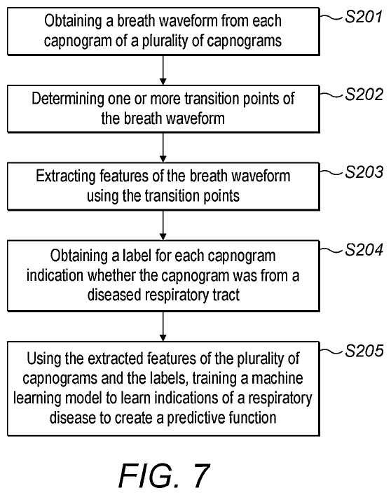

FIG. 7 shows a method for training a machine learning model to learn indicators of a capnogram being produced from a user with a cardiorespiratory disease.

In step S201, a breath waveform is obtained from each capnogram of a plurality of capnograms. Step S201 mirrors step S101 by splitting a capnogram into sections consisting of a single, full breath waveform. Using a larger quantity of data with a wider variety of properties to train a machine learning model typically leads to a better trained model capable of making more accurate predictions. Preferably, the greater quantity of data is provided by obtaining breath waveforms from a greater number of different capnograms (i.e. most preferably different capnograms produced at different times by different respiratory tracts), however a plurality of breath waveforms may also be obtained from a single capnogram of the plurality of capnograms.

In step S202, one or more transition points of the waveform are determined. Step S202 corresponds to step S102 described above and may be performed using the same techniques. This also includes, for example, determining the first order differential of a breath waveform, and identifying and accounting for hump artefact(s).

In step S203, features of the breath waveform are extracted using the one or more transition points determined in step S202. Step S203 corresponds to step S103 described above and may be performed using the same techniques.

When repeating steps S202 and S203 for each of the different breath waveforms of the plurality of capnograms, it is not required for the same transition point(s) to be determined for each breath waveform and/or for the same features to be extracted from every waveform. However, it is preferred that the same transition point(s) are determined and the same features extracted for a plurality of breath waveforms (and more preferably, for most of the breath waveforms) in order to more effectively train the machine learning model.

In step S204, a label is obtained for each capnogram, where the label indicates whether the capnogram was from a diseased respiratory tract (or a healthy respiratory tract). Preferably, when the label indicates that the capnogram was from a diseased respiratory tract it also indicates the cardiorespiratory disease that was affecting the tract at the time the capnogram was recorded. For example, when training a machine learning model to learn indicators of a capnogram being produced from a user with COPD, it is preferable that the capnograms are labelled

“COPD” or “non-COPD”, rather than “COPD” or “healthy”, as a respiratory tract without COPD may have another cardiorespiratory disease that affects the waveform(s) of the produced capnogram.

It will be apparent that step S204 may be performed before or after any of steps S201, S202, and S203.

In step S205, the labels and the extracted features of the plurality of capnograms are used to train a machine learning model to learn indicators of a cardiorespiratory disease to create a predictive function. The resulting predictive function is a trained machine learning model suitable for use in step S104.

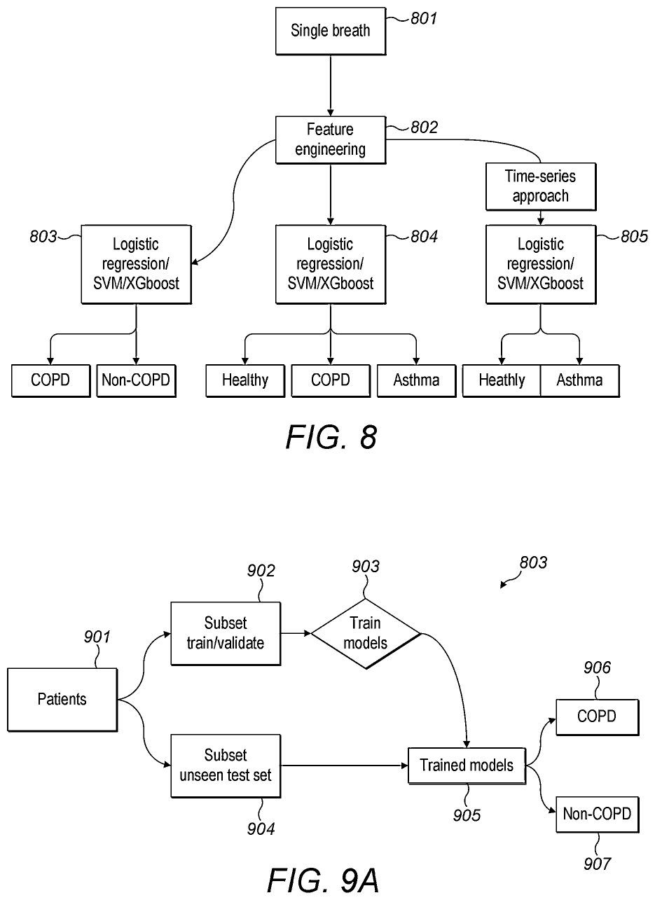

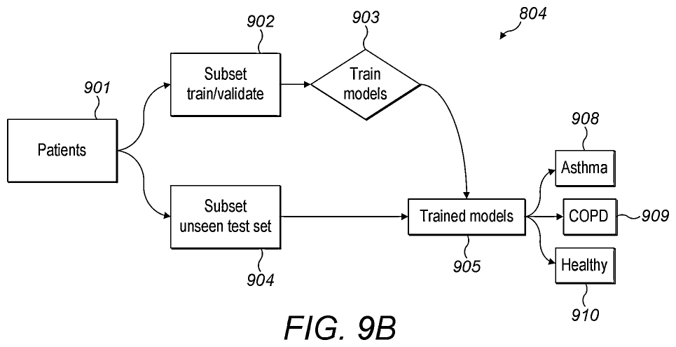

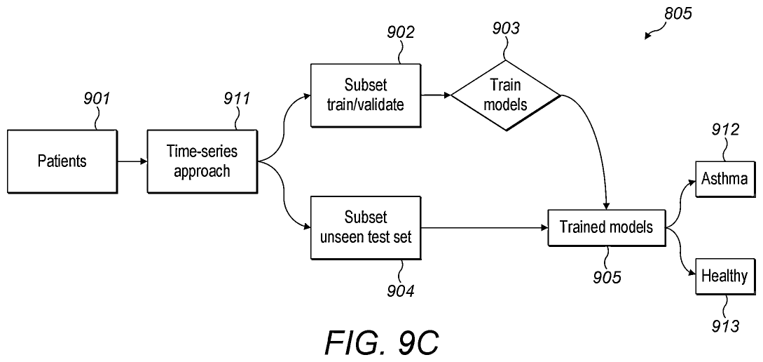

In preferred examples, multiple machine learning approaches may be combined as illustrated schematically in FIG. 8. FIGS. 9A to 9C illustrate the three components of FIG. 8.

Based on a representation of a single breath waveform, including for example an average waveform or templated waveform, 801, feature engineering 802 takes place as set out below. Multiple machine learning models and then used in three example components 803, 804, 805 of a system.

Three different ML models were selected in this example, namely logistic regression (LR), gradient boosted decision trees (XGBoost), and the support vector machine (SVM). LR and XGBoost were chosen as simple but effective and interpretable algorithms, while the SVM, and more specifically the kernel SVM, were included to classify on a higher-dimensional, non-linear feature space. Deep learning methods could also be used however in these examples may not be preferred so as to maintain interpretability and to avoid escalation of computational complexity. It will be understood that deep learning such as an ANN or a BiLSTM may also be applicable for use with the present methods and in particular with the present feature engineering.

FIG. 9A illustrates a schematic for the implementation of identifying COPD patients from a single representation of a breath record. The patients 901 are subset into training and validation data sets 902 upon which models are trained 903. Optionally, the capnograms are obtained and grouped according to a patient stratified k-fold with each patient's recordings existing within only a single fold of this splitting. A further subset 904 can optionally be used as an unseen test set input to the trained models 905 learned to classify COPD 906 or non-COPD 907.

Most of the state-of-the-art methods focus on the classification accuracy without providing tangible/explainable information that could be useful to clinicians or experts. Else, other products provide only basic information such as the end-tidal CO2 and respiratory rate which doesn't provide enough information to differentiate between various diseases. Existing methods usually aim to classify between COPD and normal; most likely patients are showing symptoms of lung disease if they are being checked up, hence they are more likely to suffer from another disease, for that reason it is preferred that models are trained on COPD vs Non-COPD as opposed to normal/healthy.

FIG. 9B illustrates how the classification task can be extended to a multi-class classifier, for example a three-class classifier. The three classes are COPD 909, Asthma 908 and Healthy 910. The same methods and features used in the binary task of COPD vs NON-COPD are employed.

FIG. 9C illustrates a further extension in which the task may be extended using a time-series approach to examine changes in features over time.

Asthma patients are observed to be more heterogeneous, with some severe asthma patients observed to have capnography waveform geometries closer to COPD, and others closer to healthy individuals. This is not something that has been specifically targeted previously as it has been difficult to access longitudinal capnography data for many different patients, and the accuracy and speed of alternative sensors are not high enough to resolve fast transitions as effectively. Trying to identify asthma patients from single metrics would be very challenging. The present methods are able to capture this longitudinal information.

Longitudinal information may include multiple breath records from the same capture sessions, or multiple capnograms captured over time. A time-series approach is introduced, calculating the variability over an n day window where the variability is defined as the standard deviation between all the features extracted. Hence, the third system classifies between healthy and asthma patients. As shown in FIG. 9C data is captured from the patients over a period of time. As indicated above, in examples, features extracted from each waveform at each time may be compared, for example to identify mean, median or standard deviation of the features or alternatively a statistical distribution over time of the features. These may be used to train the models further based on the variability of the features.

As shown in FIG. 9C, the patients 901 undergo a time-series approach 911 before being subset into training and validation data sets and unseen test data sets. The trained models 905, once trained 903, then classify patient data into asthma and healthy.

Above, an algorithmic approach to identifying the transition points of the capnogram S102 has been described. In an alternative implementation, the transition points may be identified S102 using a machine learning approach.

To train a model, a plurality of breath waveforms may be obtained. As above, this may be in the form of a set of discrete samples. A set of labels may be applied to one or more samples of each breath waveform. The labels may represent the features that the machine learning model is intended to learn. For example, the label may correspond to a phase of the particular breath waveform. Alternatively, labels may correspond to a positive or negative indication of whether a sample corresponds to a transition point. Alternatively, labels may correspond to a real number that indicates how far along the breath a particular transition point occurs. In a preferred example, there may be five output classes, the labels each identifying to which of the five classes the sample belongs.

The labels and set of training breath waveforms may each be provided to a classifier or other suitable machine learning model, such as a convolutional neural network, which may be configured to learn to predict the transition points for an unseen set of samples. In a particular example, a logistic regression model can be trained in a supervised manner on the defined labels and samples so as to learn to classify each individual sample into the set of defined output classes. The input may be a whole breath standardised to a fixed length. Similarly, there may be a plurality of logistic regression models, one for each sample.

In an implementation phase, a set of unseen samples of a breath may be provided to a model (or a plurality of models in the example above where there is a trained model for each sample) trained on the above labels and training breath waveforms. The unseen samples are classified into the defined output classes. Based on the samples that neighbour the borders of each class, the transition points between phases of the unseen waveform can be identified. This information can then be used to extract features of the waveform, as identified elsewhere in this disclosure.

### Disease Classification

It was described above how a classifier or other machine learning model may be trained to identify the likelihood of a cardiorespiratory disease being associated with an unseen breath waveform or capnogram. It will be understood that while we might describe the concepts here using the terminology of supervised learning, the concepts are equally applicable to unsupervised learning. As an example, we describe classification but it would be understood that clustering techniques could also be used.

In a specific example, a higher-complexity deep learning model may be able to detect features in CO₂ waveforms, beyond a more explainable approach that relies on feature engineering. In a specific example, a 1D convolutional neural-network may be trained on standardised CO2 waveforms. Saliency maps, a method to assist in interpretability, may be applied to identify the regions of the waveform that contribute most to predicting a likelihood of COPD.

Despite this, supervised machine learning and interpretable methods have important advantages in the healthcare space.

The model proposed may be trained and configured to identify a likelihood of a capnogram being associated with a disease, or a subtype of a disease. In specific examples, the disease is a cardiorespiratory disease, specifically COPD, Asthma, Asthma-COPD Overlap Syndrome (ACOS) or a subtype of a cardiorespiratory disease, such as asthma with small airways disease. In the description below, we refer generally to disease but we also mean subtypes. For example, where we refer to disease A and disease B, we also mean disease A and subtype B or subtype A and subtype B, etc.

To predict a likelihood of a disease from a capnogram, the machine learning model may be configured as a classifier with the model being trained on labels indicating that the breath waveform or capnogram is clinically associated with a disease, or a subtype of a disease, or not, as the case may be. In this example, the model may be configured as a binary classifier trained to predict values in a range [0,1], where 0 would correspond to no disease and 1 to a certainty of disease being present. In other words, each label belongs to a class {disease, not_disease}.

From this likelihood, it is proposed to classify a risk of disease based on a stratification model. It is contemplated to apply a set of thresholds (e.g. 4) to stratify into the likelihood into a set of classes (e.g. 5) to assist with clinical rule-in/rule-out and to allow a risk-based approach to be used for integrating into a clinical pathway.

In another example, the model may be configured, not as a binary classifier for disease or no disease, but instead to identify a likelihood of different diseases. Here, the machine learning model may be configured as a classification problem identifying a prediction between disease A and disease B. The model may be trained on labels belonging to classes indicating that the obtained data is associated with a respective disease. In this example, the model may be configured as a binary classifier trained to predict values in a range [0,1], where 0 would correspond to disease A and 1 to disease B. In other words, each label belongs to a class {disease A, disease B}. Similar to the above, those likelihood (or probability) values may be stratified to identify a risk of each disease.

A specific example of this approach is presented in the context of Asthma and Asthma-COPD Overlap Syndrome (ACOS). Note, it was identified above that time-series data may be used for certain diseases and features based on that time-series data may be used for training. In this example, labels are used belonging to classes Asthma and ACOS, with a binary classifier trained to output a likelihood of Asthma or ACOS.

According to the Dutch Hypothesis, asthma and airway hyper-responsiveness predispose patients to COPD. This has been supported by research showing asthmatics have a 10× higher risk of developing chronic bronchitis and a 17× higher risk of developing emphysema than non-asthmatics. An early and accurate diagnosis of ACOS in asthmatics is essential to introduce COPD medications, such as long-acting muscarinic receptor antagonists, which can alleviate symptoms and slow irreversible airway remodeling.

Spirometry is the current gold-standard for diagnosing ACOS but is technique-dependent, non-specific, and requires administration by a trained healthcare professional. This, along with a lack of precise clinical criteria for ACOS, results in significant under and misdiagnosis. There is therefore a need for a simple, safe, reliable, and precise diagnostic test to identify when asthmatics have progressed to ACOS.

In this specific example, a model (e.g. a logistic regression model) may be trained on features derived from capnograms of Asthma and ACOS participants from clinical studies. Performance may be measured on an unseen test set of Asthma, and ACOS participants. A likely clinical use for this model is to rule in or rule out a diagnosis of ACOS in Asthma patients where the model is most confident. The output may be stratified into Highly likely Asthma; Likely Asthma; Indeterminate; Likely ACOS; or Highly Likely ACOS, based on threshold values of the probability value output by the model. In this example, waveform features driving classification are related to the alpha angle region, which represents the transition of alveolar gas to larger airways. These features correlated with FEV1/FVC, supporting their hypothesized role as markers of increased obstruction.

In an additional specific example of this concept, ACOS may also be detected in a binary classification context of [COPD; Asthma]. In this example, the model may be trained on features from the N-tidal capnogram, as described above, as well as additional clinical features. To elaborate, the machine learning model may, for example, be trained on labels belonging to classes COPD and Asthma, i.e. {asthma, COPD}, with the model trained and configured to predict a likelihood of the capnogram being associated with COPD and Asthma and the output prediction corresponding to an overlap of the two diseases and thus indicative of a risk of both diseases being present.

It is also contemplated to extend the machine learning problem to multi-class classification, with labels belonging to a plurality of classes. Each class may correspond to a respective disease or lack thereof. This extension facilitates the prediction of a likelihood of one of those specific classes. In a specific example, the classes may comprise COPD, Asthma or ACOS (or subtypes e.g. asthma, chronic bronchitis subtype of COPD, emphysema subtype of COPD) such that the likelihood value output can be used by clinicians to aid a diagnosis of three similar and overlapping outcomes, reducing the risk of misdiagnosis.

The implication from the above description is that each approach may be effective using a single label classification, that is each capnogram may be assigned one (and only one) discrete label. However, this may not necessarily be the case. For example, the problems may be extended to multi-label classification such as {asthma, not_COPD} or other similar multi-label problems, depending on the clinical input to the data obtained alongside the capnogram. Similarly, the multi-label classification may also predict a non-disease association alongside the disease association.

Each of these techniques may be applied to the same capnogram or breath waveform and either the individual outputs presented, or the output combined or weighted to produce a clinical indication.

In a further example of techniques disclosed herein, the techniques may be applied to quantify small airways obstruction in asthma using the fast-response capnometer and interpretable machine learning. Asthma is a major noncommunicable disease that inflames the conducting zone of the bronchial tree, and is the most common chronic disease among children. It affected an estimated 262 million people in 2019—causing 455,000 deaths—and amounts to $50 billion annually in direct healthcare costs in the USA alone. Small airways disease is a poorly understood contributory factor to severe asthma, targeting the non-cartilaginous 8th and higher generation of the tracheobronchial tree. It has been demonstrated to triple the odds of systemic corticosteroid use, and increase the odds of acute exacerbation 6 fold. The prevailing measure of small airways obstruction is an abnormally low % predicted forced expiratory flow rate between 25% and 75% of vital capacity (% pred. FEF 25-75%) as measured during spirometry. Using the techniques proposed it is possible to use a capnometer to assess the degree of small airways disease as measured by % pred. FEF 25-75%.

Capnograms may be drawn from studies that recruit asthmatic participants. Participants may be asked to breathe normally into the N-Tidal capnometer at rest for 75 seconds at a time. These capnography signals may then be denoised, split into individual breath waveforms, and featurised into geometrical features for each capnogram. An explainable machine learning classifier (XGBoost) may then be trained on the features from the capnograms of a perecentage (e.g. 82%) of the patients to distinguish % pred. FEF 25-75% <50% from ≥50%, and tested on capnograms from each of the remaining patients.

### Severity Estimation

Above it has been described how a breath waveform may be classified into a set of output classes. Additionally and alternatively, that classification may comprise a measure of the severity of the respiratory disease such as COPD, referred to here as a severity metric or severity index. In other words, the severity index may be calculated as part of the output classification, however, within this terminology we intend that a specific classification may not be output, instead the model may be configured to output the severity index within the meaning of the phrase output classification.

For the classification of respiratory diseases, such as COPD, machine learning algorithms may be trained and evaluated on COPD and non-COPD participants including COPD patients ranging from a set of differently defined severity. That is, each breath waveform may have a defined severity and the waveforms may be grouped by their severity. Severity estimation may be achieved by training an additional model (e.g. logistic regression) to distinguish mild from very severe COPD with an output probability interpreted as disease severity. Models may be evaluated using five-fold nested cross-validation with unseen test patients.

Note, although COPD is used here as an example, the techniques proposed are applicable to other diseases similarly. For example, the techniques could be applied with Asthma and GINA score, or similarly with other diseases.

COPD severity may be derived from the % predicted FEV1 of a subset of COPD patients where spirometry was available.

In pulmonary function testing, a post-bronchodilator FEV1/FVC ratio of <0.7 is commonly considered diagnostic for COPD. The Global Initiative for Chronic Obstructive Lung Disease (GOLD) system categorises airflow limitation into stages. In patients with FEV1/FVC <0.7: GOLD 1—mild: FEV1 ≥80% predicted; GOLD 2—moderate: 50% ≤FEV1 <80% predicted; GOLD 3—severe: 30% ≤FEV1 <50% predicted; GOLD 4—very severe: FEV1 <30% predicted. Where we use the term mild we may refer to a GOLD 1 category and severe may refer to a GOLD 4 category. Accordingly, it may be possible using techniques proposed to predict a categorisation of an unseen waveform into these categories alongside or instead of a metric quantifying severity. Similarly, machine learning models may be trained using breath waveforms allocated into these categories.

In a particular implementation example, model training and evaluation may be undertaken using a nested cross-validation scheme. A full training dataset may be first split into five “outer-loop” folds using a group-stratified cross-validation procedure, ensuring the capnograms of a single patient would not be split across outer-loop training and test sets. For each outer-loop iteration, the outer-loop training set may be re-split using group-stratified cross-validation into training and validation sets. An “inner-loop” of five training/validation iterations may then be run on the split outer-loop training set to find a set of hyperparameters which optimised performance. The full outer-loop training set may then be subsequently used to train a model using optimised hyperparameters, which can be tested on the unseen outer-loop test set. This process may be repeated once for each of five different outer-loop test sets and the results of the optimised models on all outer-loop test sets aggregated to produce the results of the overall model performance.

The performance variability across the five outer-loop test sets gives a measure of model generalisability.

The nested cross-validation scheme of training and evaluation has two distinct advantages. First, it maximises the data that was tested on as every patient was placed exactly once in an unseen outer-loop test set, which is especially important for the severity estimation task where there may be a small number of patients in the dataset. Second, nested cross-validation mitigates the risk of optimistic model bias caused by tuning hyperparameters and testing the optimised model on the same test set.

As indicated, it is proposed to provide severity estimation by training a machine learning classifier to distinguish between participants with different severities of respiratory disease, e.g. mild COPD or severe COPD, and use the probability of having severe disease outputted by this classifier as an indication of COPD severity.

This ‘severity index’ would indicate where on the spectrum between mild and very severe COPD a patient falls. When testing on unseen patients of all severities, the output probability distribution for patients between mild and severe would be expected to fall in between the distributions for mild and severe, and for a patient's probability output (severity index) to steadily increase as their disease progresses.

Where there is a small number of patients available for this analysis and the predominance of comorbidities especially among mild patients, analysis may be conducted after removing comorbid patients from the dataset. This is so the machine learning model would be able to more effectively infer the underlying signal of COPD severity without the potential confound of comorbidities.

Classification may be performed using logistic regression in this analysis due to its simplicity, explainability and since logistic regression has been shown in COPD classification tasks to be as accurate as non-linear models.

An objective has been to apply interpretable machine learning techniques to capnography data and evaluate the performance of the diagnostic and severity classifiers. An aim is to construct a classifier that can distinguish capnograms of patients with varying severities of COPD from those without COPD. A secondary aim is to develop an alternative severity index to % predicted FEV1 which could be used as an aid by clinicians to quantify COPD severity.

In summary, techniques presented herein facilitate real-time geometric waveform analysis and machine-learning-based classification to diagnose all severities of different respiratory diseases, such as COPD. In contrast to commonly used ‘black box’ machine learning methodologies, a set of highly explainable methods can be used that can provide traceability for machine diagnosis back to individual geometric features of the pCO2 waveform and their associated physiological properties suggestive of obstructive airways disease. In addition, the probability output of a separate machine learning model could be used as an alternative severity index for COPD, with the interpretability of the implemented machine learning techniques providing a picture of COPD patients' capnographic progression from mild through severe.

### Average Waveform

Another way to extract features from one or more capnograms produced from a user is to use a plurality of breath waveforms to generate a single, smooth, average breath waveform from which features can then be extracted. The extraction of features from this average breath waveform can be carried out instead of or in addition to the extraction of features from individual breath waveforms.

First, a plurality of breath waveforms from one or more capnograms produced from the user are obtained. While these breath waveforms could be provided as inputs for the method, in many instances the method will involve processing the one or more capnograms to obtain the plurality of breath waveforms. This will typically involve separating the one or more incoming capnograms into a plurality of capnogram sections. The capnogram sections will preferably each represent a single breath waveform corresponding to a single respiratory cycle and the method will therefore involve determining the delta and gamma transition points for each capnogram section in any of the manners described above, with the portion of the capnogram section between the delta transition point and the gamma transition point then extracted to generate a breath waveform. Alternatively, the cut-off points could be varied to capture more of the baseline, which can allow for more information relating to a user's respiratory cycle to be captured. It is also possible for the breath waveform to capture just a portion of a capnogram produced from the user, such that the breath waveform captures a portion of a respiratory cycle rather than a whole respiratory cycle.

The plurality of breath waveforms will typically be of different durations, reflecting the fact that a user's respiratory cycle will vary in length from one breath to another. Likewise, the CO₂ respired will vary between respiratory cycles, leading to different breath waveforms having different amplitudes. Since these differences between the breath waveforms of a single user will often not be indicative of a cardiorespiratory disease, it is preferable for the average breath waveform not to reflect the differences in duration of the plurality of breath waveforms and, optionally, the differences in amplitude of the plurality of breath waveforms. Instead, in embodiments of the present invention the average breath waveform is used to represent the average shape of a breath waveform of the user.

To this end, the duration and, optionally, amplitude of the plurality of breath waveforms are normalised to generate a plurality of normalised breath waveforms. By duration of a breath waveform we mean the time from the start of a respiratory cycle and the end of a respiratory cycle. As will be discussed below, the start and end points can be determined in different ways, but in all cases these points are selected consistently across all breath waveforms. When normalising, the duration of each of the plurality of breath waveforms is scaled such that the duration is the same for all breath waveforms.

As with the normalisation of the duration of the plurality of breath waveforms, the amplitude of the plurality of breath waveforms may be optionally scaled such that the amplitude is the same for all breath waveforms. This amplitude can be determined in different ways, and could be as simple as the difference in CO₂ readings between the highest and lowest points of a breath waveform. In the ideal case, the highest CO₂ reading will be the end-tidal CO₂ value, and this will indeed be the case for many breath waveforms. However, the maximum amplitude will often be found at a different point of a breath waveform, either due to measurement errors or to underlying conditions of the corresponding respiratory cycle. This can lead to inconsistency in how the breath waveforms are normalised, so it is preferable to use the end-tidal CO₂ value to normalise the breath waveforms. This involves extracting an end-tidal CO₂ value from each breath waveform in any of the manners described above (e.g., determining the beta transition point) and then adjusting the amplitude of each of the plurality of breath waveforms such that each of the plurality of breath waveforms has the same end-tidal CO2 value.

Once the breath waveforms have been normalised, an average breath waveform is generated from the plurality of normalised breath waveforms, preferably using a generalised additive model (GAM). While other averaging methods, such as taking the geometric mean or the median of the plurality of breath waveforms, are possible, using a GAM allows a more physiologically accurate waveform to be generated which is not overfitted to the data from the plurality of breath waveforms. It is also more resilient to outliers in this data.

Another advantage, which will be discussed in more detail with respect to the specific example set out below, is that typically the plurality of breath waveforms each comprise a series of data points. Simply taking the geometric mean or median of the data points from the plurality of breath waveforms would therefore lead to an average breath waveform which itself comprised a discontinuous set of data points. In contrast, the use of a GAM leads to a functional representation of a breath which is continuous, thereby allowing for the average breath waveform to be used at any resolution desired by an operator.

Nevertheless, in some embodiments of the invention other methods are used to generate the average waveform.

Features can then be extracted from the average breath waveform in a similar manner to how features are extracted from a single breath waveform, and a trained machine learning model can be applied to these features to predict a cardiorespiratory disease. A machine learning model can be employed which makes use of features extracted from individual breath waveforms of the user when predicting a cardiorespiratory disease in addition to the features extracted from the average breath waveform. Alternatively, a machine learning model can be employed which makes use of features extracted from either individual breath waveforms of the user or the average breath waveform alone when predicting a cardiorespiratory disease.

The usefulness of the average breath waveform generated, whether this has been generated using a GAM or using some other method, can be improved further still by first identifying whether any of the plurality of normalised breath waveforms are anomalous and then excluding said anomalous normalised breath waveforms before generating the average breath waveform.

One advantageous way to identify anomalous normalised breath waveforms is to interpolate the normalised breath waveforms to all have the same number of data points, and to directly compare the data points of one normalised breath waveform with the data points from the other normalised breath waveforms. Any normalised breath waveforms having one or more anomalous data points are then identified as being anomalous and excluded.

This approach has been found to be less computationally intensive than other approaches to excluding anomalous breaths. For example, interpolating the normalised breath waveforms and comparing data points to identify anomalous breaths has been found to be less computationally intensive than standard anomaly detection algorithms.

A specific example of this method will now be described which has been found to be particularly advantageous.

This example has been described in the case of breath waveforms obtained from a single capnogram, but it could equally be applied in the case of breath waveforms obtained from multiple capnograms. Likewise, the skilled person will understand that other modifications of this specific example are possible. For example, the example described below could be modified to use breath waveforms capturing different portions of a respiratory cycle than the waveform between the delta transition point and the gamma transition point, such as those which capture more of the baseline or those which capture a predefined portion of a respiratory cycle rather than a whole respiratory cycle.

Firstly, a capnogram is obtained and then separated into M constituent capnogram segments, whereafter only the waveform between each of the delta transition point and the gamma transition point is extracted for each capnogram segment. These transition points may be identified in any of the manners discussed above, for example such that the waveform is extracted between the delta angle start and the gamma angle end for each capnogram segment. The use of the delta and gamma transition points is especially advantageous as this results in an average breath waveform that is more similar to a single breath waveform extracted from a capnogram. As such, features that can be extracted from a single breath waveform may also be extracted from an average breath waveform. Furthermore, in embodiments in which a machine learning model is to be employed which makes use of features extracted from individual breath waveforms of the user when predicting a cardiorespiratory disease in addition to the features extracted from the average breath waveform, these transition points may have already been identified for one or more of the breath waveforms.

The breath waveforms are numbered from 1 to M, with breath waveform m being defined as the pair (tₘ, yₘ), where tₘ is the the vector of timestamps for each CO₂ partial pressure value in the vector yₘ and having length Nₘ. All breaths are then individually normalised to the same duration and optionally height (which is to be understood as meaning the same as amplitude), whilst maintaining its unique shape. The optional height normalisation may be achieved by scaling the end-tidal CO₂ (which may be calculated in any of the manners described above) value to 5 KPa, so that

$${\tilde{y}}_{m} = \frac{5}{{y}_{m , end - tidal}} {y}_{m} ∀ m ∈ \{ 1 , … , M \} .$$

The time series points are shifted such that they begin at 0, and are then scaled linearly such that the final point is at 3 seconds,

$${\tilde{t}}_{m} = \frac{3}{{t}_{m , {N}_{m}}} ( {t}_{m} - {t}_{m , 1} ) ∀ m ∈ \{ 1 , … , M \} .$$

The skilled person will of course understand that other values of the scaled end-tidal CO₂ value could be chosen, as could other values of the scaled duration. As has been discussed above, while optional, it is advantageous in some instances to exclude anomalous normalised breath waveforms. In the present example, this is done by linearly interpolating all (normalised) breath waveforms to the same length (which is to say to all comprise the same number of data points), to give (t̅ₘ, y̅ₘ) Vm E {1, ... , M} and |t̅ₘ|=|y̅ₘ|=N: =400 ∀ m∈{1, . . . , M}. The skilled person will understand that other values of N could be chosen. This allows the nth data point of each (normalised) breath waveform to be directly compared with the nth data point of every other (normalised) breath waveform. Breath waveforms for which one or more of these data points are found to be significant outliers can then be excluded as being anomalous.

In the present example, any breath waveforms m∈{1, . . . , M} having at least one data point n∈{1, . . . , N} for which y̅_{m,n} is further than 3 standard deviations from the median (i.e. the median of the nth data points of every breath waveform) are excluded, leaving a subset of the original set of normalised breath waveforms, namely M̅. The skilled person will understand that a different threshold for abnormal breath waveforms could be chosen. For example, a different number of standard deviations could be chosen as the threshold, or alternatively a different measure could be chosen, such as a percentage difference from the median.

Using this set of (t̅ₘ, y̅ₘ)∀m∈M̅, a generalised additive model (GAM) is then used to generate a single, smooth, average breath for the capnogram. In this specific example, the GAM is defined as E[y|t]=f (t), which aims to minimise

$$\frac{1}{N \vert \overline{M} \vert} {\sum }_{n = 1}^{N} {\sum }_{m ∈ \overline{M}} {( {\overline{y}}_{m , n} - f ( {\overline{t}}_{m , n} ) )}^{2} + λ {\int }_{t = 0}^{3} {{f}^{″} ( t )}^{2} dt$$

The final term provides a cost for excessive curvature, and the parameter λ therefore controls the level of penalisation due to excessive curvature, thereby limiting the degree of overfitting. The skilled person will understand that this term is optional and may be omitted by setting λ=0. Although other processes such as Gaussian Process Regression or kernel ridge regression could be used, these are more computationally intensive or produce less smooth curves than a GAM and take a long time to fit for high resolution average breath waveforms.

This function to be minimised can be rewritten by concatenating all the (t̅ₘ, y̅ₘ)∀m∈M̅ into a single set (t̅, y̅) where each of the vectors are of length |t̅|=|y̅|=N|M̅|, and minimising

$$\frac{1}{N \vert \overline{M} \vert} {\sum }_{i = 1}^{N \vert \overline{M} \vert} {( {\overline{y}}_{i} - f ( {\overline{t}}_{i} ) )}^{2} + λ {\int }_{t = 0}^{3} {{f}^{″} ( t )}^{2} dt$$

This model is fitted by learning the transformation function f, using the set of breaths (t̅, y̅).

In order to fit the model, a class of functions from which f should belong is first chosen. In the present case, this is the set of cubic splines. These are the set of piecewise cubic polynomial functions that interpolate between specified control points (knots) while matching the zeroth, first and second derivatives at these knots, forming a single, smooth, fit with continuity down to the second derivative (as shown in figure). The cubic spline is built up using a linear combination of the set of basis-splines, Δⱼ (t), to give

$$f ( t ) = {\sum }_{j = 1}^{J} {β}_{j} {ϕ}_{j} ( t )$$

where the coefficients, βⱼ, are fitted by the minimisation and Δⱼ (t). The specific form of Δⱼ (t) are known in the art, and are distributed uniformly in time.

The number J is specified, where the first and last basis functions are positioned at the first and last times, such that they can all be scaled horizontally to fit within the 0 to 3 second window. (As noted above, the breath waveforms could be normalised to different durations, in which case the basis functions would also be scaled to a different duration.) The coefficients are fitted with Poisson Iteratively Reweighted Least Squares (PIRLS) iterations. It has been found that J=100 allows any kind of breath (i.e. healthy or non-healthy) of any length to be fitted well. The skilled person of course understands, however, that different fitting and optimisation procedures could be used.

The final numerical average waveform output is yₙ=f (tₙ) where tₙ is a series of time points in the range [(t̅), (t̅)], which in this example means

$${t}_{n} = 3 \frac{n - 1}{N - 1} ∀ n ∈ \{ 1 , … , N = 400 \} .$$

(As noted above, a different value of N could be chosen.) Using this average breath waveform, features may be calculated using the methods described above.

The advantage of using splines is that this approach is more computationally efficient than other approaches, for example fitting a high-order polynomial.

Likewise, cubic basis splines have been found to represent the best trade-off between ensuring continuity of the GAM up to the second order derivative and computational complexity. Lower order splines will be discontinuous at the second (and possibly also first) derivative, and this discontinuity in curvature reduces the effectiveness of feature extraction since some features are based on the curvature parameters of the average breath waveform. Higher order splines do not have this issue, but they increase the computational complexity in fitting the GAM and can also lead to oscillations in the average waveform which do not reflect the underlying data.

One alternative is to use Bezier curves instead of splines. However, using Bezier curves without the use of knots is significantly more computationally intensive, while conversely using knots with Bezier curves will lead to discontinuities in the first derivative. Both of these are significant disadvantages as compared with the present method.

As has been noted, other modifications to the example given above are possible. One modification relates to how the breaths are normalised.

While the duration and height of the breath waveforms may be normalised to arbitrary values, in some preferable embodiments the breaths are normalised to the average end-tidal CO₂ value of the series of breath waveforms, with the average end-tidal CO₂ value defined as follows:

$${y}_{end - tidal} := \frac{1}{M} {\sum }_{m = 1}^{M} {y}_{m , end - tidal}$$

This leads to the following normalised height for each breath waveform:

$${\tilde{y}}_{m} = \frac{{y}_{end - tidal}}{{y}_{m , end - tidal}} {y}_{m}$$

Similarly, the duration of each breath waveform may be normalised to the average duration of the series of breath waveforms. This is particularly advantageous when the normalisation step is combined with extraction of the breath waveforms from their respective capnogram segments, rather than receiving the breath waveforms after this extraction has occurred.

In this approach, the timestamp of the delta angle, t_{m,d}, and the timestamp of the gamma angle t_{m,g} are identified for each breath waveform, with the difference between these Δtₘ=t_{m,g}−t_{m,d} defining the length of the portion of the breath waveform to be used when generating the average breath waveform. The average of these values is then taken to determine an average delta time, an average gamma time, and an average difference:

$${t}_{d} := \frac{1}{M} {\sum }_{m = 1}^{M} {t}_{m , d} \\ {t}_{g} := \frac{1}{M} {\sum }_{m = 1}^{M} {t}_{m , g} \\ Δ {t}_{μ} := \frac{1}{M} {\sum }_{m = 1}^{M} Δ {t}_{m}$$

(Although the arithmetic mean has been used as the average, other measures of the mean could be used, as could the median.)

The timestamps of each of the breath waveforms is then translated as follows:

$${t}_{m} := \frac{Δ {t}_{μ}}{Δ {t}_{m}} ( {t}_{m} - {t}_{m , d} ) + {t}_{d} .$$

Here, the first term translates and rescales the breath waveform so that the delta transition point is at 0 seconds and the time difference between the delta and gamma transition points equal to the average Δt_{μ}. The second term then translates the breath back so that the delta transition point for this breath is at the timestamp of the average delta transition point. (As noted above, the breath waveforms can be normalised to any arbitrary value, such that Δt_{μ} =3 for a length of 3 s).

At this point, the delta transition points and gamma transition points are synced between the breath waveforms. However, each breath waveform will typically comprise a portion of the baseline, and the amount of this baseline will vary between the breath waveforms. This means that the start and end times of the breath waveforms will differ. In order to address this, the breaths are cut to a predefined length. This can be arbitrary, but to extract breaths between the delta transition point and the gamma transition point this preferably involves the following steps.

First, the average duration of the breath from the gamma transition point to the breath end is identified. For example, where the arithmetic mean is used to define the average difference:

$$Δ {t}_{g} := \frac{1}{M} {\sum }_{m = 1}^{M} {t}_{m , {N}_{m}} - {t}_{m , g}$$

where t_{m,N}_{ₘ} is the timestamp of the end point of breath m.

Each breath waveform is then cropped at its closest timestamp to t_{g}+Δt_{g} to define (\tilde{t}ₘ, \tilde{y}ₘ) where, as before, |\tilde{t}ₘ=|\tilde{y}ₘ=Ñₘ. (Alternatively, each breath waveform could be cropped to the average breath duration such that (when using

$${\tilde{t}}_{m , {N}_{m}} = \frac{1}{M} {\sum }_{m = 1}^{M} {t}_{{N}_{m}} ∀ m ∈ \{ 1 , … , M \} . )$$

Any breaths that are smaller than the chosen cropping length are extended by either copying their final value at regular intervals until the cropping length, or by a linear fit until the chosen cropping length.

Another modification to the example given above relates how to the optional step of excluding anomalous breaths is performed. In some embodiments the breath waveforms are interpolated to the same length without excluding anomalous breaths. In other embodiments no interpolation is performed and nor are anomalous breaths excluded. In those embodiments where the breath waveforms are interpolated and anomalous breaths excluded, it can be advantageous to revert the non-excluded breaths back to their original non-interpolated form after the exclusion of anomalous breaths.

The generation of the average waveform may also vary from the approach described above. While it is advantageous to use a GAM for this, this need not use splines to learn the transformation function f. For example, linear and factor terms could also be used. While it has been found that linear terms on their own do not typically produce a good fit, factor terms lead to a better fit, although this fit is typically more jagged than when using splines. Any combination of spline terms, factor terms, or linear terms could therefore be used.

For example, when including these additional terms the function fitted by the GAM is

$$f ( t ) = {\sum }_{j = 1}^{J} {β}_{j} {ϕ}_{j} ( t ) + {\sum }_{j = 1}^{{J}_{α}} {α}_{j} {φ}_{j} ( t ) + {\sum }_{j = 1}^{{J}_{γ}} {γ}_{j} {ψ}_{j} ( t )$$

where aⱼ for j∈{1, . . . , J_{α}} and γⱼ for j∈{1, . . . , J_{γ}} are coefficients and J, J_{α}, J_{β} are arbitrary values selected when fitting the transformation function f. The basis functions of the factor terms, ϕⱼ , and the basis functions for the linear terms, ψⱼ, will of course be known to the skilled reader. Typically |βⱼ|>>|αₖ|∀j∈{1, . . . , J_{α}, k∈{}1, . . . , J_{α}} and |βⱼ|>>|γₖ|∀j∈{1, . . . , J_{α}, k∈{}1, . . . , J_{γ}}, i.e. the weights assigned to spline terms are much greater than for the linear or factor terms, due to the better fitting provided by the spline terms.

It is also possible to generate the average waveform without using a GAM.

One such example generates an average waveform using the average of the CO2 values at each time point. For example, when using an arithmetic mean:

$${y}_{i}^{*} = \frac{1}{\vert \overline{M} \vert} {\sum }_{m = 1}^{\overline{M}} {\overline{y}}_{m , i} ∀ i ∈ \{ 1 , … , N \}$$

and, furthermore, a standard deviation can be calculated at each time point:

$${σ}_{i}^{*} = \sqrt{\frac{1}{\vert \overline{M} \vert} {\sum }_{m = 1}^{\overline{M}} {( {\overline{y}}_{m , i} - \frac{1}{\vert \overline{M} \vert} {\sum }_{m = 1}^{\overline{M}} {\overline{y}}_{m , i} )}^{2}} ∀ i ∈ \{ 1 , … , N \}$$

As mentioned above, although they are more computationally intensive or produce less smooth curves than a GAM, two other possible approaches are Gaussian process regression and kernel ridge regression.

Similar to the GAM approach, these approaches start with a pre-processing of the received breaths to arrive at breath waveforms (t̅, y̅). Kernel ridge regression then aims to find the weight vector α that minimises

$${\sum }_{i = 1}^{\vert \overline{t} \vert} L ( {y}_{i} , {( K α )}_{i} ) + λ {α}^{T} K α$$

where L is a loss function and K is the kernel matrix Kᵢⱼ=k(t̅, y̅) built using the kernel function, k(x, y), where for example

$$k ( x , y ; σ ) = {e}^{- \frac{{‖ x - y ‖}_{2}^{2}}{2 {σ}^{2}}}$$

is the RBF kernel. Similar to the GAM implementation, λ acts as a smoothing parameter that may be set for when an optimisation algorithm tunes α.

Then, the average waveform is generated using yₙ=Σᵢ₌₁^{|ē|}k(t̅ᵢ, tₙ)αᵢ where tₙ is a series of time points in the range [(t̅), (t̅)].

After preprocessing of the received breaths to arrive at breath waveforms (t̅, y̅), Gaussian process regression starts by defining the new time points of the average waveform as a series of time points in the range [(t̅), (t̅)], that are referred to as t* in the following.

The Gaussian Process Regressor model will be known to the skilled person, and in this implementation makes use of a kernel function specified similarly to the kernel ridge regression model that will form kernel matrices whose elements are defined as

Kᵢⱼ=k(t̅ᵢ, t̅ⱼ)

K_{i*j}=k(t̅_{j*}, t̅ᵢ)

K_{i*j*}=k(t̅_{i*}, t̅_{j*})

Using a hyperparameter σ_{ε}, the average waveform is predicted as

y*=K*(K+σ_{ε}²I)⁻¹y̅

and the standard deviations given by the vector

σ*={square root over (diag(K**=K*(L+σ_{ε}²I)⁻¹K*))}

(where the square root is element-wise) and I is the identity matrix.

An extra optional but preferable step is to automatically select hyperparameters which are those of the kernel function and σ_{ε}. This is done by minimising the negative log marginal likelihood using a standard optimisation algorithm:

$$p ( y \vert X ) = \frac{1}{2} {{\overline{y}}^{T} ( K + {σ}_{ε}^{2} I )}^{- 1} \overline{y} + \frac{1}{2} \vert K + {σ}_{ε}^{2} I \vert + \frac{n}{2} 2 π$$

The hyperparameters found from this minimisation can then be inserted into the calculations above.

A variation of this Gaussian process regression framework is described in is known as sparse Gaussian process regression and, although this variation sacrifices a degree of accuracy, is more computationally efficient.

After obtaining (t̅, y̅), ‘inducing inputs’ are then specified. These inducing inputs are an additional series of t values that are smaller in size than t̅. We name these t′. A set of kernel matrices are then defined using the elements of the inducting inputs:

K′ᵢⱼ=k(t̅ᵢ, t̅′ⱼ)

Kᵢⱼ^{′′}=k(t̅′ᵢ, t̅′̅ⱼ)

Kᵢⱼ^{′*}=k(t̅′ᵢ, t̅*ⱼ)

Further distribution parameters are also defined as follows:

$$\sum = {( {K}^{″} + \frac{1}{{σ}_{ε}^{2}} {K}^{′ T} {K}^{′} )}^{- 1} \\ μ = \frac{1}{{σ}_{ε}^{2}} {K}^{″} \sum {K}^{′ T} \overline{y} \\ A = {K}^{″} \sum {K}^{″}$$

These parameters allow

$${y}^{*} = {K}^{′ * T} {K}^{{″}^{- 1}} μ \\ {σ}^{*} = \sqrt{diag ( {K}^{**} - {K}^{′ * T} {K}^{{″}^{- 1}} {K}^{′ *} + {K}^{{′}^{* T}} {K}^{{″}^{- 1}} {AK}^{{″}^{- 1}} {K}^{′ *} )}$$

to be computed.

(Note that the inducing inputs t′ can be chosen manually or alternatively selected automatically using an optimisation algorithm that minimises the following expression with respect to t′:

$$\frac{1}{2 {σ}_{ε}^{2}} Tr [ K - Q ] - N ( \overline{y} ; 0 , {σ}_{ε}^{2} I + Q )$$

where Q=K′K″⁻¹K′^{T}, N is a normal distribution and Tr is the trace function.)

A final alternative to using a GAM is a similar process to Gaussian process regression but which models each point in time as a “Student t” distribution rather than a Gaussian distribution.

After obtaining (t̅, y̅), a kernel function is now defined as before but with a small addition Σ_{ε}²I as follows:

\tilde{k}(x, y)=k(x, y)+Σ_{ε}²I

This gives kernel matrices defined by the elements as

\tilde{K}ᵢⱼ=\tilde{k}(t̅ᵢ, t̅ⱼ)

\tilde{K}*ᵢⱼ=\tilde{k}(t*ᵢ, t̅ⱼ)

\tilde{K}ᵢⱼ^{**}=\tilde{k}(t̅*ᵢ, t̅*ⱼ)

This gives us the equations to calculate the average waveform and its standard deviation as:

$${y}^{*} = {\tilde{K}}^{{*}^{T}} {\tilde{K}}^{- 1} \overline{y} \\ {σ}^{*} = \sqrt{diag ( \frac{ω + {\overline{y}}^{T} {\tilde{K}}^{- 1} \underline{y}}{v + \vert \overline{t} \vert - 2} ( {\tilde{K}}^{**} - {\tilde{K}}^{{*}^{T}} {\tilde{K}}^{- 1} {\tilde{K}}^{*} ) )}$$

where ω, v are hyperparameters. As before, the hyperparameters, including any from the kernel function can first be chosen manually or optimised by minimising the negative log marginal likelihood:

$$\frac{v + \vert \overline{t} \vert}{2} \vert \tilde{K} + ω \overline{y} {\overline{y}}^{T} \vert - \frac{v + \vert \overline{t} \vert - 1}{2} \vert \tilde{K} \vert + {Γ}_{\vert \overline{t} \vert} ( \frac{v + \vert \overline{t} \vert - 1}{2} ) - {Γ}_{\vert \overline{t} \vert} ( \frac{v + \vert \overline{t} \vert}{2} ) + \frac{\vert \overline{t} \vert}{2} \vert πω \vert$$

$$where {Γ}_{\vert \overline{t} \vert} ( x ) ≡ {π}^{\frac{\vert \overline{t} \vert ( \vert \overline{t} \vert - 1 )}{4}} {\prod }_{i = 1}^{\vert \overline{t} \vert} Γ ( x + \frac{1}{2} - \frac{i}{2} )$$

and Γ is the gamma function.

Any or all steps of the methodology may be implemented in a remote or cloud computing device or locally at the edge, i.e. on a device capable of retrieving the capnogram data. In examples, training is performed centrally before the implantation or test phase is performed using a trained model stored locally either on the device, e.g. with the model parameters stored in memory on SoC, or on a local computer. The trained model and its associated parameters may be stored centrally, i.e. on the cloud. Methods and processes described herein can be embodied as code (e.g., software code) and/or data. The models, methodologies and algorithms may be implemented in hardware or software as is well-known in the art of machine learning. For example, hardware acceleration using a specifically programmed Graphical Processing Unit (GPU) or a specifically designed Field Programmable Gate Array (FPGA) may provide certain efficiencies. For completeness, such code and data can be stored on one or more computer-readable media, which may include any device or medium that can store code and/or data for use by a computer system. When a computer system reads and executes the code and/or data stored on a computer-readable medium, the computer system performs the methods and processes embodied as data structures and code stored within the computer-readable storage medium. In certain embodiments, one or more of the steps of the methods and processes described herein can be performed by a processor (e.g., a processor of a computer system or data storage system).

Generally, any of the functionality described in this text or illustrated in the figures can be implemented using software, firmware (e.g., fixed logic circuitry), programmable or nonprogrammable hardware, or a combination of these implementations. The terms “component” or “function” as used herein generally represents software, firmware, hardware or a combination of these. For instance, in the case of a software implementation, the terms “component” or “function” may refer to program code that performs specified tasks when executed on a processing device or devices. The illustrated separation of components and functions into distinct units may reflect any actual or conceptual physical grouping and allocation of such software and/or hardware and tasks.

1. A method for classifying a capnogram, the method comprising:
obtaining a breath waveform from a capnogram;
determining one or more transition points of the breath waveform, wherein the transition points comprise:
a delta transition point between an expiratory baseline and an expiratory upstroke,
a gamma transition point between an inspiratory downstroke and an inspiratory baseline, and
an alpha transition point between the expiratory upstroke and an expiratory plateau;
extracting features of the breath waveform using the transition points; and
applying a trained machine learning model to the extracted features, wherein the trained machine learning model is configured to output a classification of the capnogram from the features.

2. A method for training a machine learning model to learn indicators of a capnogram, the method comprising, for a plurality of capnograms:
obtaining a breath waveform from each capnogram;
determining one or more transition points of the breath waveform, wherein the transition points comprise:
a delta transition point between an expiratory baseline and an expiratory upstroke,
a gamma transition point between an inspiratory downstroke and an inspiratory baseline, and
an alpha transition point between the expiratory upstroke and an expiratory plateau;
extracting features of the breath waveform using the one or more transition points;
obtaining a label for each capnogram indicating a capnogram classification and,
using the extracted features of the plurality of breath waveforms and the corresponding labels of the respective capnograms, training a machine learning model to learn indicators of a capnogram to create a classifying function.

3. The method of claim 1, wherein the breath waveform represents a single respiratory cycle; and
optionally, the obtaining the breath waveform comprises splitting the capnogram into a plurality of capnogram sections, wherein each capnogram section represents a single breath waveform corresponding to the single respiratory cycle.

4. The method of claim 1, wherein the plurality of transition points further comprise a beta transition point between the expiratory plateau and the inspiratory downstroke.

5. The method of claim 1, wherein determining the one or more transition points comprises determining a derivative of the breath waveform; optionally,
optionally, the derivative of the breath waveform is a first order differential of the breath waveform; and

6. The method of claim 5, wherein the method further comprises:
using the first order differential of the breath waveform to determine whether the breath waveform is an anomalous breath waveform; and,
when the breath waveform is an anomalous breath waveform, rejecting the breath waveform.

7. The method of claim 5, wherein determining the first order differential of the breath waveform comprises applying a time-based smoothing filter to the breath waveform.

8. The method of claim 5, wherein determining the plurality of transition points comprises:
identifying a hump artefact in the breath waveform and, when there is a hump artefact, accounting for the hump artefact during the determining of the one or more transition points;
optionally, identifying the hump artefact comprises:
performing peak detection to identify local minima of the breath waveform;
identifying prominent minima from the local minima;
identifying the maximum value of the breath waveform and/or determining the beta transition point;
dividing the breath waveform into a first section not including the maximum value of the breath waveform, and a second section including the maximum value of the breath waveform and/or the beta transition point;
when at least one prominent minimum is identified, searching for hump artefact(s) in the first section of the breath waveform; and/or
when no prominent minima are identified, using the first order differential of the breath waveform to search for hump artefact(s) in the first section of the breath waveform.

9. The method of claim 1, wherein determining the transition points comprises determining the beta transition point, wherein determining the beta transition point comprises:
performing peak detection to identify local maxima of the breath waveform;
identifying prominent maxima from the local maxima;
when only a single prominent maximum is identified, determining this as a beta transition point; and,
when a plurality of prominent maxima are identified, determining the most prominent maximum and defining this as the beta transition point.

10. The method of claim 1, wherein determining the transition points comprises determining the delta transition point, wherein determining the delta transition point comprises:
determining the first point in time at which a first order differential of the breath waveform is above a delta threshold; and
defining the first point as the delta transition point.

11. The method of claim 1, wherein determining the transition points comprises determining the gamma transition point, wherein determining the gamma transition point comprises:
identifying the minimum value of a first order differential of the breath waveform; and,
defining the gamma transition point as the first point in time after the minimum value at which the first order differential of the breath waveform is higher than a gamma threshold.

12. The method of claim 1, wherein determining the transition points comprises determining the alpha transition point, wherein determining the alpha transition point comprises:
identifying the maximum value of a first order differential of the breath waveform;
identifying the maximum value of the breath waveform and/or determining the beta transition point; and
defining the alpha transition point as the first point in time after the maximum value of the first order differential, between the maximum value of the first order differential and the maximum value of the breath waveform and/or the beta transition point, at which the first order differential of the breath waveform is less than an alpha threshold;
optionally, determining the alpha transition point further comprises:
when no point between the maximum value of the first order differential of the breath waveform and the maximum value of the breath waveform is less than the alpha threshold, or when no point between the maximum value of the first order differential of the breath waveform and the beta transition point is less than the alpha threshold, increasing the alpha threshold.

13. The method of claim 1, wherein determining the transition points comprises determining an alpha transition point, wherein determining the alpha transition point comprises:
calculating a line between the delta transition point and the maximum value of the breath waveform, or calculating a line between the delta transition point and the beta transition point; and,
defining the alpha transition point based on the distance between the breath waveform and the calculated line.

14. The method of claim 1, wherein determining the transition points comprises:
applying a trained machine learning model to a set of discrete samples of the breath waveform, the breath waveform representing a whole breath, wherein the machine learning model is configured to classify each sample into one of a plurality of output classes, each class representing a region of the breath waveform, and wherein the machine learning model is trained by:
obtaining a label associated with each discrete sample of a plurality of breath waveforms, each breath waveform being represented by a set of samples representing a whole breath and each label indicating which of a plurality of output classes that sample corresponds to; and,
training the machine learning model on the labels and the samples to learn to classify a sample of a set of samples representing a whole breath into a class of the plurality of output classes.

15. The method of claim 1, further comprising:
extracting features from a plurality of breath waveforms recorded from the same user;
determining the variability of the extracted features;
wherein the variability of the extracted features is also used to apply the trained machine learning model to classify the capnogram, or wherein the variability of the extracted features is also used to train the machine learning model to create the classifying function;
optionally, the plurality of breath waveforms are recorded from the same user over a time period comprising two or more days.

16. The method of claim 1, wherein the machine learning model comprises at least one of: logistic regression, a gradient boosting decision tree, a support-vector machine, AdaBoost, and a random forest.

17. The method of claim 1, wherein the trained machine learning model is further configured to output an indication of the importance of an extracted feature that led to the classification of the capnogram.

18. The method of claim 1, wherein extracting features of the breath waveform using the transition point(s) comprises determining an angle of the transition point(s).

19. The method of claim 18, wherein determining the angle of the transition point(s) comprises:
fitting a first linear function and a second linear function to the adjacent phases on either side of the transition point and measuring the angle between the first and second linear functions; and/or
fitting a third linear function to the expiratory upstroke or the inspiratory downstroke and measuring the angle between the third linear function and the horizontal.

20. The method of claim 1, wherein extracting features of the breath waveform using the transition point(s) comprises fitting a quadratic function to the expiratory plateau, and determining a coefficient of the quadratic function.

21. The method of claim 1, wherein extracting features of the breath waveform using the transition point(s) comprises fitting a hyperbolic tangent function to the expiratory upstroke and/or the inspiratory downstroke, and determining a coefficient of the hyperbolic tangent function(s).

22. The method of claim 1, wherein the classification of the capnogram comprises a probability value corresponding to a severity of a respiratory disease;
optionally, the machine learning model is trained based on capnograms labelled with a respective severity of a respiratory disease.

23. A method of diagnosing a cardiorespiratory disease, the method comprising the method of claim 1, and further comprising:
diagnosing the presence of a cardiorespiratory disease.

24. The method of claim 1, further comprising obtaining a capnogram from a user, wherein the capnogram comprises a breath waveform.

25. The method of claim 1, wherein the machine learning model is trained and configured to predict a likelihood that the capnogram is associated with a cardio respiratory disease;
optionally, the machine learning model is configured to output the likelihood that the capnogram is associated with the cardiorespiratory disease.

26. The method of claim 1, wherein the machine learning model is trained using labels belonging to a class representing that a capnogram is associated with a cardiorespiratory disease;
optionally, the labels further belong to a plurality of classes, each class representing that capnogram is associated with a cardiorespiratory disease, each class corresponding to a respective disease;
optionally, the labels further belong to a class representing that a capnogram is not associated with a cardiorespiratory disease; and
optionally, the cardiorespiratory disease is selected from a group comprising: COPD, Asthma, Asthma-COPD Overlap Syndrome (ACOS), small airways disease, chronic bronchitis subtype of COPD and emphysema subtype of COPD.

27. The method of claim 25, further comprising stratifying the output by comparing the likelihood to a set of threshold values, each strata representing a risk of the capnogram being associated with the cardiorespiratory disease.

28. The method according to claim 2, wherein the classifying function is configured to predict a severity of a respiratory disease.

29. An apparatus configured to perform the method of claim 1.

30. A computer readable medium comprising instructions which, when executed by a processor, cause the processor to perform the method of claim 1.
